(19) United States
(12) Patent Application Publication
(10) Pub. No.: US 2023/0333469 A1
(43) Pub. Date: Oct. 19, 2023

(54) FILM FORMING COMPOSITION FOR LITHOGRAPHY, RESIST PATTERN FORMATION METHOD, AND CIRCUIT PATTERN FORMATION METHOD

(71) Applicant: Mitsubishi Gas Chemical Company, Inc., Tokyo (JP)

(72) Inventors: Takashi SATO, Hiratsuka-shi, Kanagawa (JP); Tadashi OMATSU, Hiratsuka-shi, Kanagawa (JP); Takashi MAKINOSHIMA, Hiratsuka-shi, Kanagawa (JP); Masatoshi ECHIGO, Tokyo (JP)

(21) Appl. No.: 18/014,130

(22) PCT Filed: Jul. 8, 2021
(86) PCT No.: PCT/JP2021/025746

§ 371 Date: Dec. 30, 2022

(30) Foreign Application Priority Data
Jul. 8, 2020 (JP) 2020-118023
Aug. 7, 2020 (JP) 2020-135055

(51) Int. Cl. G03F 7/004; G03F 7/038; G03F 7/039; G03F 7/11; C09D 133/14; H01L 21/027

(52) U.S. Cl. CPC G03F 7/0045; C09D 133/14; G03F 7/038; G03F 7/039; G03F 7/11; H01L 21/0275

(57) ABSTRACT

The present invention provides a film forming composition for lithography containing at least one selected from a compound having a predetermined structure and a resin obtained using the compound as a monomer.

### TECHNICAL FIELD

The present invention relates to a film forming composition for lithography, a resist pattern formation method, and a circuit pattern formation method.

### BACKGROUND ART

In the production of semiconductor devices, fine processing is practiced by lithography using photoresist materials. In recent years, further miniaturization based on pattern rules has been demanded along with increase in integration and speed of LSIs (large scale integrated circuits). Light source for lithography used upon forming resist patterns has been shifted to ArF excimer laser (193 nm) having a shorter wavelength from KrF excimer laser (248 nm). Introduction of extreme ultraviolet (EUV, 13.5 nm) is also expected.

However, because conventional polymer-based resist materials have a molecular weight as large as about 10,000 to 100,000 and also wide molecular weight distribution, in lithography using such a polymer-based resist material, roughness occurs on a pattern surface; the pattern dimension becomes difficult to be controlled; and there is a limitation in miniaturization. Accordingly, various low molecular weight resist materials have been proposed so far in order to provide resist patterns having higher resolution. The low molecular weight resist materials are expected to provide resist patterns having high resolution and small roughness, because of their small molecular sizes.

Various materials are currently known as such low molecular weight resist materials. For example, an alkaline development type negative type radiation-sensitive composition (see, for example, Patent Literature 1 and Patent Literature 2) using a low molecular weight polynuclear polyphenolic compound as a main component has been suggested; and as a candidate of a low molecular weight resist material having high heat resistance, an alkaline development type negative type radiation-sensitive composition (see, for example, Patent Literature 3 and Non Patent Literature 1) using a low molecular weight cyclic polyphenolic compound as a main component has been suggested as well. Also, as a base compound of a resist material, a polyphenolic compound is known to be capable of imparting high heat resistance despite a low molecular weight and useful for improving the resolution and roughness of a resist pattern (see, for example, Non Patent Literature 2).

In addition, in Patent Literature 4, a resist composition containing a compound having a specific structure and an organic solvent has been proposed as a material that is excellent in etching resistance and is also soluble in a solvent and applicable to a wet process.

Also, as the miniaturization of resist patterns proceeds, a problem of resolution (a problem that suitable patterns cannot be formed due to low resolution) or a problem of collapse of resist patterns after development (a problem that resist patterns collapse (or wave) due to low resistance to pattern collapse during development if the resist stiffness is low) arises. Therefore, thinner resist films have been desired. However, if resist films are merely made thinner, it is difficult to obtain the film thicknesses of resist patterns sufficient for substrate processing. Therefore, there has been a need for a process of preparing an underlayer film between a resist and a semiconductor substrate to be processed, and imparting, also to this underlayer film, functions as a mask for substrate processing in addition to a resist pattern. In addition, processes have been put to practical use that increase the etching resistance of the resist to reduce the required film thickness, or that transfers the resist pattern to an underlayer film between the resist and the semiconductor substrate to be processed, and provide this underlayer film with high etching resistance and photosynthesis functions.

Various underlayer films for such lithography are currently known. For example, as a material for realizing resist underlayer films having the selectivity of a dry etching rate close to that of resists, unlike conventional underlayer films having a fast etching rate, an underlayer film forming material for a multilayer resist process containing a resin component having at least a substituent that generates a sulfonic acid residue by eliminating a terminal group under application of predetermined energy, and a solvent has been suggested (see, for example, Patent Literature 5). Also, in order to realize an underlayer film for lithography having the selectivity of a dry etching rate smaller than that of resists, an underlayer film material comprising a polymer having a specific repeat unit has been suggested (see, for example, Patent Literature 6). Furthermore, as a material for realizing underlayer films for lithography having the selectivity of a dry etching rate slower than that of semiconductor substrates, a resist underlayer film material comprising a polymer prepared by copolymerizing a repeat unit of an acenaphthylene and a repeat unit having a substituted or unsubstituted hydroxy group has been suggested (see, for example, Patent Literature 7).

Meanwhile, as materials having high etching resistance for this kind of resist underlayer film, amorphous carbon underlayer films formed by chemical vapor deposition (CVD) using methane gas, ethane gas, acetylene gas, or the like as a raw material are well known. However, resist underlayer film materials that can form resist underlayer films by a wet process such as spin coating or screen printing have been demanded from the viewpoint of processability.

In addition, Patent Literature 8 describes an underlayer film forming material for lithography containing a compound having a specific structure as a material that is excellent in etching resistance, has high heat resistance, and is soluble in a solvent and applicable to a wet process.

As for methods for forming an intermediate layer used in the formation of a resist underlayer film in a three-layer process, for example, a method for forming a silicon nitride film (see, for example, Patent Literature 9) and a CVD formation method for a silicon nitride film (see, for example, Patent Literature 10) are known. Also, as intermediate layer materials for a three-layer process, materials comprising a silsesquioxane-based silicon compound are known (see, for example, Patent Literature 11 and Patent Literature 12).

### CITATION LIST

### Patent Literature

Patent Literature 1: Japanese Patent Laid-Open No. 2005-326838
Patent Literature 2: Japanese Patent Laid-Open No. 2008-145539
Patent Literature 3: Japanese Patent Laid-Open No. 2009-173623
Patent Literature 4: International Publication No. WO 2013/024778
Patent Literature 5: Japanese Patent Laid-Open No. 2004-177668
Patent Literature 6: Japanese Patent Laid-Open No. 2004-271838
Patent Literature 7: Japanese Patent Laid-Open No. 2005-250434
Patent Literature 8: International Publication No. WO 2013/024779
Patent Literature 9: Japanese Patent Laid-Open No. 2002-334869
Patent Literature 10: International Publication No. WO 2004/066377
Patent Literature 11: Japanese Patent Laid-Open No. 2007-226170
Patent Literature 12: Japanese Patent Laid-Open No. 2007-226204

### Non Patent Literature

Non Patent Literature 1: T. Nakayama, M. Nomura, K. Haga, M. Ueda: Bull. Chem. Soc. Jpn., 71, 2979 (1998)
Non Patent Literature 2: Shinji Okazaki et al., “New Trends of Photoresists,” CMC Publishing Co., Ltd., September 2009, pp. 211-259

### SUMMARY OF INVENTION

### Technical Problem

However, the materials described in Patent Literatures 1 to 12 and Non Patent Literatures 1 to 2 still have room for improvement as film forming materials for lithography from the viewpoint of simultaneously satisfying high levels of solubility in organic solvents, heat resistance, etching resistance, and resist pattern formability. In addition, there is also room for improvement as film forming materials for lithography from the viewpoint of satisfying a good balance of solubility in organic solvents, storage stability and thin film formability, etching resistance, sensitivity, and resist pattern formability at high levels.

The present invention has been made in view of the problems of the prior art described above, and an object of the present invention is to provide a film forming composition for lithography that is useful for forming a lithography film, a resist pattern formation method, and a circuit pattern formation method.

### Solution to Problem

The present inventors have, as a result of devoted examinations to solve the problems described above, found out that a composition containing a compound having a specific structure is useful for forming a lithography film, leading to completion of the present invention.

Specifically, the present invention includes following aspects.

A film forming composition for lithography comprising at least one selected from: a compound represented by general formula (1); a compound represented by general formula (3); a compound represented by formula (4); a compound represented by formula (5); and a resin obtained using the compound represented by formula (4) or the compound represented by formula (5) as a monomer,

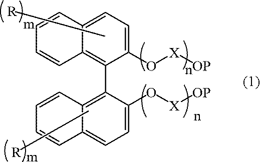

wherein
R each independently represents an aromatic group having 6 to 36 carbon atoms and optionally having a substituent or a heteroatom;
X each independently represents an alkanediyl group having 2 to 4 carbon atoms or alkanediylcarbonyl group having 1 to 4 carbon atoms, each of which optionally has a substituent;
P each independently represents an alkyl group having 1 to 30 carbon atoms, aryl group having 6 to 30 carbon atoms, alkenyl group having 2 to 20 carbon atoms, or alkynyl group having 2 to 20 carbon atoms, each of which optionally has a substituent, or a hydrogen atom, a crosslinkable group, or a dissociable group; and
m each independently represents an integer of 1 to 6 and n each independently represents an integer of 0 to 4,

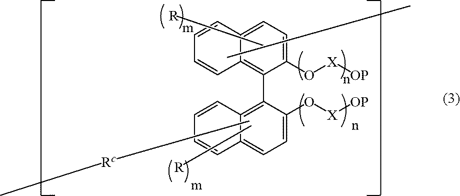

wherein
   R each independently represents an aromatic group having 6 to 36 carbon atoms and optionally having a substituent or a heteroatom;
   R^{c} each independently represents a single bond, a linear or branched alkylene group having 1 to 20 carbon atoms and optionally having a substituent, or an arylene group having 1 to 20 carbon atoms and optionally having a substituent;
   X each independently represents an alkanediyl group having 2 to 4 carbon atoms or alkanediylcarbonyl group having 1 to 4 carbon atoms, each of which optionally has a substituent;
   P each independently represents an alkyl group having 1 to 30 carbon atoms, aryl group having 6 to 30 carbon atoms, alkenyl group having 2 to 20 carbon atoms, or alkynyl group having 2 to 20 carbon atoms, each of which optionally has a substituent, or a hydrogen atom, a crosslinkable group, or a dissociable group; and
   m each independently represents an integer of 1 to 6 and n each independently represents an integer of 0 to 4, 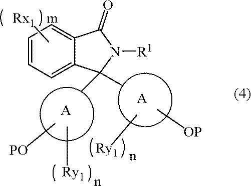
   wherein
      A each independently represents an aromatic group having 6 to 10 carbon atoms;
      P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group;
      R¹ represents an aromatic substituent having 6 to 10 carbon atoms or an alkyl group having 1 to 20 carbon atoms;
      Rx₁ each independently represents a hydrogen atom, an alkyl group having 1 to 20 carbon atoms, an alkoxy group having 1 to 20 carbon atoms, or a halogen;
      Ry₁ each independently represents a hydrogen atom, an alkyl group having 1 to 20 carbon atoms, an alkoxy group having 1 to 20 carbon atoms, or a halogen; and
      m represents an integer of 0 to 4 and n represents an integer of 0 to 4, and 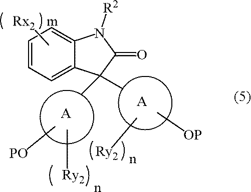
      wherein
         A each independently represents an aromatic group having 6 to 10 carbon atoms;
         P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group;
         R² represents an aromatic substituent having 6 to 10 carbon atoms or an alkyl group having 1 to 20 carbon atoms;
         Rx₂ each independently represents a hydrogen atom, an alkyl group having 1 to 20 carbon atoms, an alkoxy group having 1 to 20 carbon atoms, or a halogen;
         Ry₂ each independently represents a hydrogen atom, an alkyl group having 1 to 20 carbon atoms, an alkoxy group having 1 to 20 carbon atoms, or a halogen; and
         m represents an integer of 0 to 4 and n represents an integer of 0 to 4.

The film forming composition for lithography according to [1], wherein the compound represented by general formula (1) is a compound represented by general formula (2),

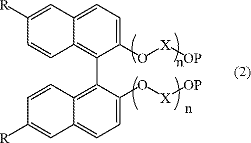

wherein R, X, P, and n are the same as defined in formula (1).

The film forming composition for lithography according to [1], comprising the compound represented by general formula (3).

The film forming composition for lithography according to [1], comprising at least one selected from: the compound represented by formula (4); the compound represented by formula (5); and the resin obtained using the compound represented by formula (4) or the compound represented by formula (5) as a monomer.

The film forming composition for lithography according to [4], wherein
the compound represented by formula (4) is a compound represented by formula (6); and
the compound represented by formula (5) is a compound represented by formula (7),

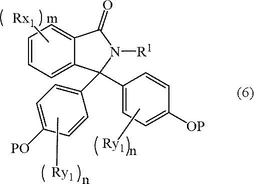

wherein
   P, R¹, Rx₁ Ry₁, m, and n are the same as defined in formula (4), and 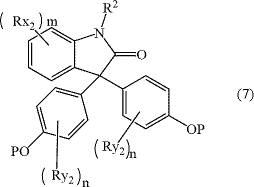
   wherein
      P, R², Rx₂ Ry₂, m, and n are the same as defined in formula (5).

The film forming composition for lithography according to [4], wherein the resin is a resin represented by formula (8),

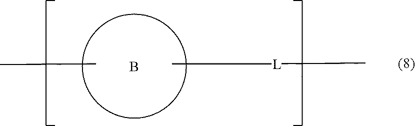

wherein
B is a constituent unit derived from the compound represented by formula (4) and/or a constituent unit derived from the compound represented by formula (5);
L is a single bond, a linear or branched alkylene group having 1 to 20 carbon atoms and optionally having a substituent, or an arylene group having 1 to 20 carbon atoms and optionally having a substituent; and
when there are a plurality of B and/or L, they are independent of each other.)

The film forming composition for lithography according to any one of [1] to [6], further comprising a solvent.

The film forming composition for lithography according to any one of [1] to [7], further comprising an acid generating agent.

The film forming composition for lithography according to any one of [1] to [8], further comprising a crosslinking agent.

A resist pattern formation method, comprising:
a photoresist layer formation step of forming a photoresist layer on a substrate using the film forming composition for lithography according to any one of [1] to [9]; and
a development step of irradiating a predetermined region of the photoresist layer with radiation for development, thereby obtaining a resist pattern.

The resist pattern formation method according to [10], wherein the resist pattern is an insulating film pattern.

A resist pattern formation method, comprising:
a resist underlayer film formation step of forming a resist underlayer film on a substrate using the film forming composition for lithography according to any one of [1] to [9];
a photoresist layer formation step of forming at least one layer of photoresist layer on the resist underlayer film; and
a development step of irradiating a predetermined region of the photoresist layer with radiation for development, thereby obtaining a resist pattern.

A circuit pattern formation method, comprising:
a resist underlayer film formation step of forming a resist underlayer film on a substrate using the film forming composition for lithography according to any one of [1] to [9];
an intermediate layer film formation step of forming an intermediate layer film on the resist underlayer film;
a photoresist layer formation step of forming at least one layer of photoresist layer on the intermediate layer film;
a resist pattern formation step of irradiating a predetermined region of the photoresist layer formed through the photoresist layer formation step with radiation for development, thereby obtaining a resist pattern;
an intermediate layer film pattern formation step of etching the intermediate layer film with the resist pattern as a mask, thereby obtaining an intermediate layer film pattern;
a resist underlayer film pattern formation step of etching the resist underlayer film with the intermediate layer film pattern as a mask, thereby obtaining a resist underlayer film pattern; and
a substrate pattern formation step of etching the substrate with the resist underlayer film pattern as a mask, thereby obtaining a substrate pattern.

### Advantageous Effects of Invention

According to the present invention, it is possible to provide a film forming composition for lithography that is useful for forming a lithography film with excellent heat resistance, etching resistance, and resist pattern formability, as well as a resist pattern formation method and a circuit pattern formation method using the same.

In addition, according to the present invention, it is possible to provide a composition that is useful as a film forming material for lithography, having high solubility in organic solvents, excellent storage stability and thin film formability, high etching resistance, high sensitivity, and excellent resist pattern formability, and satisfying a good balance of these physical properties at high levels. Also, by using this composition, it is possible to provide a resist pattern formation method and a circuit pattern formation method.

### DESCRIPTION OF EMBODIMENTS

Hereinafter, embodiments of the present invention will be described (hereinafter, also referred to as the “present embodiment”). Note that the present embodiment is given in order to illustrate the present invention. The present invention is not limited only by these present embodiments.

As used herein, even if not otherwise specified, “alkyl group” may be a linear or branched alkyl group, or may be a cyclic alkyl group, and is used in the sense of encompassing all of them. Also, even if not otherwise specified, “alkoxy group” may be a linear or branched alkoxy group, or may be a cyclic alkoxy group, and is used in the sense of encompassing all of them.

A film forming composition for lithography of the present embodiment contains at least one selected from: a compound represented by general formula (1); a compound represented by general formula (3); a compound represented by formula (4); a compound represented by formula (5); and a resin obtained using the compound represented by formula (4) or the compound represented by formula (5) as a monomer. Hereinafter, a film forming composition for lithography of the present embodiment that contains at least one selected from: a compound represented by general formula (1); and a compound represented by general formula (3) will be described as a first film forming composition for lithography, and a film forming composition for lithography of the present embodiment that contains at least one selected from: a compound represented by general formula (3); a compound represented by formula (4); a compound represented by formula (5); and a resin obtained using the compound represented by formula (4) or the compound represented by formula (5) as a monomer will be described as a second film forming composition for lithography.

### First Film Forming Composition for Lithography

The first film forming composition for lithography of the present embodiment (hereinafter, also simply referred to as “first composition” and also referred to as “composition” when not distinguished from the second film forming composition for lithography, which will be described below) contains a compound represented by formula (1) below (hereinafter, also simply referred to as “compound (1)”) and/or a compound represented by formula (3) below (hereinafter, also simply referred to as “compound (3)”).

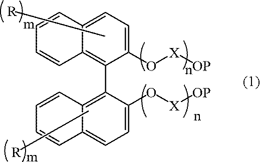

In the formula, R each independently represents an aromatic group having 6 to 36 carbon atoms and optionally having a substituent or a heteroatom.

X each independently represents an alkanediyl group having 2 to 4 carbon atoms or alkanediylcarbonyl group having 1 to 4 carbon atoms, each of which optionally has a substituent.

P each independently represents an alkyl group having 1 to 30 carbon atoms, aryl group having 6 to 30 carbon atoms, alkenyl group having 2 to 20 carbon atoms, or alkynyl group having 2 to 20 carbon atoms, each of which optionally has a substituent, or a hydrogen atom, a crosslinkable group, or a dissociable group.

m each independently represents an integer of 1 to 6.

n each independently represents an integer of 0 to 4.

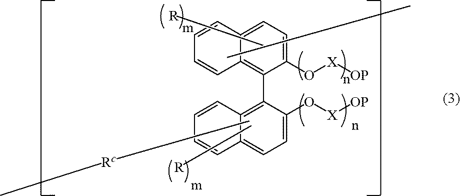

In the formula, R each independently represents an aromatic group having 6 to 36 carbon atoms and optionally having a substituent or a heteroatom.

R^{c} each independently represents a single bond, a linear or branched alkylene group having 1 to 20 carbon atoms and optionally having a substituent, or an arylene group having 1 to 20 carbon atoms and optionally having a substituent.

X each independently represents an alkanediyl group having 2 to 4 carbon atoms or alkanediylcarbonyl group having 1 to 4 carbon atoms, each of which optionally has a substituent.

P each independently represents an alkyl group having 1 to 30 carbon atoms, aryl group having 6 to 30 carbon atoms, alkenyl group having 2 to 20 carbon atoms, or alkynyl group having 2 to 20 carbon atoms, each of which optionally has a substituent, or a hydrogen atom, a crosslinkable group, or a dissociable group.

m each independently represents an integer of 1 to 6.

n each independently represents an integer of 0 to 4.

According to the first composition, it is possible to provide a film forming composition for lithography that is useful for forming a lithography film with excellent heat resistance, etching resistance, and resist pattern formability, as well as a resist pattern formation method and a circuit pattern formation method using the same.

The substituted group indicated in the compound (1) and the compound (3) of the present embodiment means a group in which one or more hydrogen atoms in a functional group have been replaced by an atom other than hydrogen atom or a functional group, unless otherwise defined. The number of substituents is not limited and may be one or more.

Examples of the substituent indicated in the compound (1) and the compound (3) of the present embodiment include, but are not limited to, a halogen atom (for example, a fluorine atom, a chlorine atom, a bromine atom, and an iodine atom), a hydroxyl group, a carboxyl group, a cyano group, a nitro group, a thiol group, a heterocyclic group, an alkyl group having 1 to 30 carbon atoms, an aryl group having 6 to 20 carbon atoms, an alkoxyl group having 1 to 30 carbon atoms, an alkenyl group having 2 to 30 carbon atoms, an alkynyl group having 2 to 30 carbon atoms, an acyl group having 1 to 30 carbon atoms, and an amino group having 0 to 30 carbon atoms.

The alkyl group described above may be in any of the following aspects: linear aliphatic hydrocarbon group, branched aliphatic hydrocarbon group, and cyclic aliphatic hydrocarbon group. In addition, the aryl group, the alkoxyl group, the alkenyl group, the alkynyl group, the acyl group, and the amino group described above may also be in any of the following forms: linear, branched, and cyclic, in the same manner as the alkyl group described above.

Since the first composition contains the compound (1) and/or the compound (3) of the present embodiment, a wet process can be applied, and heat resistance and smoothing properties are excellent. Furthermore, since the first composition contains the compound (1) and/or the compound (3), film deterioration during high temperature baking is suppressed, and a film for lithography excellent in etching resistance against oxygen plasma etching or the like can be formed. Furthermore, the first composition is also excellent in adhesiveness to a resist film and can therefore form an excellent resist pattern. Therefore, the first composition is used for film formation for lithography.

In the present embodiment, the lithography film is a generic term for films used in the lithography process, and examples thereof include an upper layer film, a resist film, an intermediate film, a resist underlayer film, an antireflection film, and a resist permanent film. Note that the upper layer film is arranged on top of the resist film and has, for example, water repellency, and as the intermediate film, those that are given various physical properties depending on their relative positional relationship with the resist film or resist underlayer film can also be employed. In addition to the above, examples of the lithography film include a film for being embedded to steps of a layer to be processed and flattening the layer. From the composition of the present embodiment, a resist film or resist underlayer film as the lithography film can be preferably formed. That is, the resist film of the present embodiment and the resist underlayer film of the present embodiment are formed from the composition of the present embodiment.

The above R preferably represents an aromatic group having 6 to 16 carbon atoms and optionally having a substituent or a heteroatom, and more preferably represents an aromatic group having 6 to 14 carbon atoms and optionally having a substituent or a heteroatom.

The above R^{c} preferably represents a single bond, a linear or branched alkylene group having 1 to 3 carbon atoms, and an arylene group having 1 to 13 carbon atoms, and more preferably represents a group selected from a methylene group, a phenylmethylene group, a biphenylmethylene group, and a cyclohexylphenylmethylene group.

The above X preferably represents an alkanediyl group having 2 to 4 carbon atoms and optionally having a substituent, more preferably an alkanediyl group having 2 to 4 carbon atoms, and still more preferably an ethanediyl group.

The above P preferably represents a hydrogen atom, a crosslinkable group, or a dissociable group.

The “crosslinkable group” in the present embodiment refers to a group that crosslinks in the presence of a catalyst or without a catalyst. Examples of the crosslinkable group include, but are not limited to, an alkyl group having 1 to 20 carbon atoms that, together with the oxygen atom to which the above P is bonded in the formula, forms an alkoxy group having 1 to 20 carbon atoms. Examples of the crosslinkable group also include a group having an allyl group, a group having a (meth)acryloyl group, a group having an epoxy (meth)acryloyl group, a group having a hydroxyl group, a group having a urethane (meth)acryloyl group, a group having a glycidyl group, a group having a vinyl-containing phenylmethyl group, a group having a group having various alkynyl groups, a group having a carbon-carbon double bond, and a group having a carbon-carbon triple bond. Furthermore, examples thereof also include a group that crosslinks in the presence of a catalyst or without a catalyst among a group containing these groups and others. The “group containing these groups” described above is preferably an alkoxy group represented by —ORx (Rx represents a group having an allyl group, a group having a (meth)acryloyl group, a group having an epoxy (meth)acryloyl group, a group having a hydroxyl group, a group having a urethane (meth)acryloyl group, a group having a glycidyl group, a group having a vinyl-containing phenylmethyl group, a group having a group having various alkynyl groups, a group having a carbon-carbon double bond, a group having a carbon-carbon triple bond, or a group containing these groups). Note that, in the present specification, for each functional group (excluding the crosslinkable group) described above as constituting the compounds of the present embodiment, if there is an overlap with the crosslinkable group, based on the presence or absence of crosslinkability, one without crosslinkability is the functional group, while one with crosslinkability is the crosslinkable group.

Examples of the alkoxy group having 1 to 20 carbon atoms include, but are not limited to, a methoxy group, an ethoxy group, a n-propoxy group, an isopropoxy group, a n-butoxy group, an isobutoxy group, a tert-butoxy group, a desoxy group, and an isocoxy group.

Examples of the group having an allyl group include a group represented by any of the following formula (X-1).

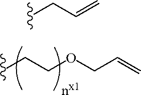

In the formula, n^{X1} represents an integer of 1 to 5.

Examples of the group having a (meth)acryloyl group include, but are not limited to, a group represented by any of the following formula (X-2).

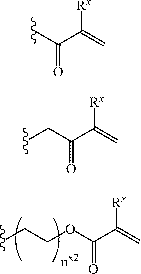

In the formula, n^{X2} represents an integer of 1 to 5.

R^{X} represents a hydrogen atom or a methyl group.

Examples of the group having an epoxy (meth)acryloyl group include, but are not limited to, a group represented by the following formula (X-3). Here, the epoxy (meth)acryloyl group refers to a group generated through a reaction between an epoxy (meth)acrylate and a hydroxyl group.

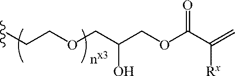

In the formula, n^{x3} represents an integer of 0 to 5, and preferably represents 0.

R^{X} represents a hydrogen atom or a methyl group, and preferably represents a methyl group.

Examples of the group having a hydroxyl group include, but are not limited to, a group represented by any of the following formula (X-5).

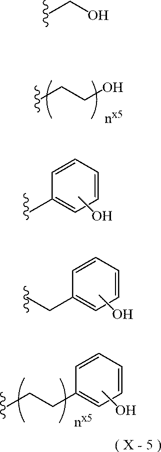

In the formula, n^{x5} represents an integer of 1 to 5, and preferably represents 1.

Examples of the group having a urethane (meth)acryloyl group include, but are not limited to, a group represented by the following formula (X-4).

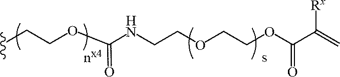

In the formula, n^{x4} represents an integer of 0 to 5, and preferably represents 0.

s represents an integer of 0 to 3, and preferably represents 0.

R^{X} represents a hydrogen atom or a methyl group and preferably represents a methyl group.

Examples of the group having a glycidyl group include, but are not limited to, a group represented by any of the following formula (X-6).

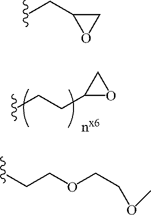

In the formula, n^{x6} represents an integer of 1 to 5.

Examples of the group having a vinyl-containing phenylmethyl group include, but are not limited to, a group represented by any of the following formula (X-7).

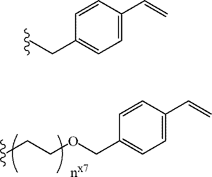

In the formula, n^{x7} represents an integer of 1 to 5, and preferably represents 1.

Examples of the group having various alkynyl groups include, but are not limited to, a group represented by any of the following formula (X-8).

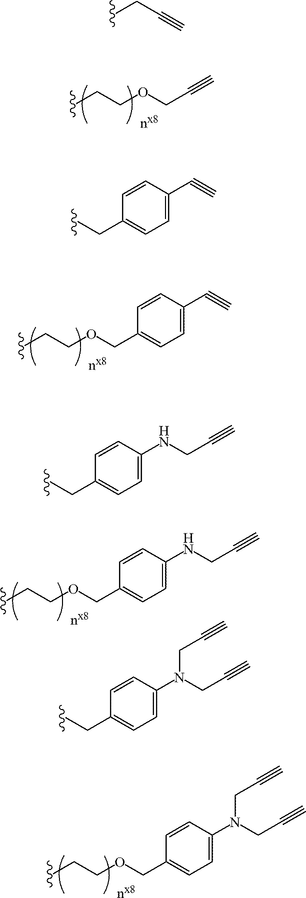

In the formula, n^{x8} represents an integer of 1 to 5.

Examples of the above carbon-carbon double bond-containing group include a (meth)acryloyl group, a substituted or unsubstituted vinylphenyl group, and a group represented by the following formula (X-9-1). In addition, examples of the above carbon-carbon triple bond-containing group include a substituted or unsubstituted ethynyl group, a substituted or unsubstituted propargyl group, a group represented by the following formula (X-9-2), and a group represented by the following formula (X-9-3).

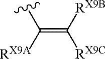

In the formula, R^{X9A}, R^{X9B} and R^{X9C} each independently represent a hydrogen atom or a monovalent hydrocarbon group having 1 to 20 carbon atoms.

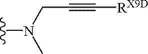

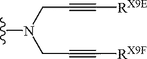

In the formulas, R^{X9D} R^{X9E} and R^{X9F} each independently represent a hydrogen atom or a monovalent hydrocarbon group having 1 to 20 carbon atoms.

The “dissociable group” in the present embodiment refers to a group that is dissociated in the presence of a catalyst or without a catalyst. Among dissociable groups, an acid dissociable group refers to a group that is cleaved in the presence of an acid to cause a change into an alkali soluble group or the like.

Examples of the alkali soluble group include, but are not limited to, a phenolic hydroxyl group, a carboxyl group, a sulfonic acid group, and a hexafluoroisopropanol group. Among them, a phenolic hydroxyl group and a carboxyl group are preferable, and a phenolic hydroxyl group is more preferable, from the viewpoint of the easy availability of an introduction reagent.

The acid dissociable group preferably has the property of causing chained cleavage reaction in the presence of an acid, for achieving pattern formation with high sensitivity and high resolution.

The acid dissociable group is not particularly limited, but can be arbitrarily selected for use from among, for example, those proposed in hydroxystyrene resins, (meth)acrylic acid resins, and the like for use in chemically amplified resist compositions for KrF or ArF.

Specific examples of the acid dissociable group can include those described in International Publication No. WO 2016/158168. Suitable examples of the acid dissociable group include a 1-substituted ethyl group, a 1-substituted n-propyl group, a 1-branched alkyl group, a silyl group, an acyl group, a 1-substituted alkoxymethyl group, a cyclic ether group, an alkoxycarbonyl group (such as —C(O)OC(CH₃)₃), and an alkoxycarbonylalkyl group (such as —(CH₂)ₙC(O)OC(CH₃)₃, wherein n = 1 to 4), which have the property of being dissociated by an acid. Note that, in the present specification, for each functional group (excluding the dissociable group) described above as constituting the compounds of the present embodiment, if there is an overlap with the dissociable group, based on the presence or absence of dissociability, one without dissociability is treated as corresponding to the functional group, and one with dissociability is treated as corresponding to the dissociable group.

Examples of the substituent for the dissociable group include, but are not limited to, a halogen atom, an alkyl group, an aryl group, an aralkyl group, an alkenyl group, an acyl group, an alkoxycarbonyl group, an alkyloyloxy group, an aryloyloxy group, a cyano group, a nitro group, and a heteroatom.

Examples of the halogen atom include, but are not limited to, a chlorine atom, a bromine atom, and an iodine atom.

The alkyl group may be linear, branched, or cyclic. Examples of the alkyl group include, but are not limited to, an alkyl group having 1 to 10 carbon atoms such as a methyl group, a tert-butyl group, a cyclohexyl group, and an adamantyl group.

Examples of the aryl group include, but are not limited to, an aryl group having 6 to 20 carbon atoms such as a phenyl group, a tolyl group, and a naphthyl group. Note that the aryl group may further have a substituent such as a halogen atom and an alkyl group having 1 to 5 carbon atoms.

Examples of the aralkyl group include, but are not limited to, a benzyl group and a phenethyl group. Note that the aralkyl group may further have a substituent such as a halogen atom and an alkyl group having 1 to 5 carbon atoms.

Examples of the alkynyl group include, but are not limited to, an ethynyl group and a propargyl group.

Examples of the acyl group include, but are not limited to, an aliphatic acyl group having 1 to 6 carbon atoms such as a formyl group and an acetyl group, and an aromatic acyl group such as a benzoyl group.

Examples of the alkoxycarbonyl group include, but are not limited to, an alkoxycarbonyl group having 2 to 5 carbon atoms such as a methoxycarbonyl group.

Examples of the alkyloyloxy group include, but are not limited to, an acetoxy group.

Examples of the aryloxy group include, but are not limited to, a benzoyloxy group.

Examples of the heteroatom include, but are not limited to, an oxygen atom, a sulfur atom, a selenium atom, a nitrogen atom, and a phosphorus atom.

A carbon atom of each group may be substituted with the heteroatom.

In the case of including the substituents described above, the number of carbon atoms in each group described in the present specification is the total number of carbon atoms including the substituents.

The above m preferably represents an integer of 1 to 4, more preferably represents an integer of 1 to 3, still more preferably represents an integer of 1 to 2, and even more preferably represents 1.

When the above m represents 1, the compound (1) is a compound represented by general formula (2) (hereinafter, also simply referred to as compound (2)).

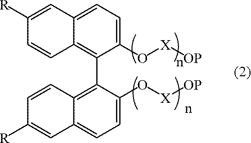

In the formula, R, X, P, and n are the same as defined in formula (1).

The above n preferably represents an integer of 0 to 3, more preferably represents an integer of 0 to 2, still more preferably represents 0 from the viewpoint of heat resistance, and still more preferably represents 1 from the viewpoint of solubility.

The compound (1) can be used directly as an underlayer film forming material for lithography. Also, the compound (1) can be used as an oligomerized resin by solely polymerizing the compound (1) by itself or by allowing the compound (1) to react with a monomer with crosslinking reactivity. Examples of the resin obtained by oligomerizing the compound (1) include the compound (3) described above. The compound (1) as the monomer used for oligomerization into the compound (3) may be one kind, or two or more kinds.

The compound (3) is obtained by solely polymerizing the compound (1) or by allowing the compound (1) to react with a compound with crosslinking reactivity.

The method for solely polymerizing the compound (1) is not particularly limited, but for example,aromatic rings of the compound (1) may be directly bonded to each other by one-electron oxidation polymerization in the presence of an oxidizing agent. In this case, R^{c} in formula (3) represents a single bond.

Examples of the above oxidizing agent include, but are not limited to, a metal salt or metal complex containing copper, manganese, iron, cobalt, ruthenium, chromium, palladium, or the like; a peroxide such as hydrogen peroxide and a perchlorate; and an organic peroxide. Among these, the metal salt or metal complex containing copper, manganese, iron, or cobalt is preferable.

The metal contained in the metal salt, such as copper, manganese, iron, cobalt, ruthenium, chromium, or palladium may function as the oxidizing agent by reduction in the reaction system.

The compound with crosslinking reactivity may be any compound as long as it can oligomerize or polymerize the compound (1), and examples thereof include an aldehyde, a ketone, a carboxylic acid, a carboxylic acid halide, a halogen-containing compound, an amino compound, an imino compound, an isocyanate compound, and an unsaturated hydrocarbon group-containing compound.

Examples of the compound (3) include, but are not limited to, a resin that has been made novolac obtained through, for example, a condensation reaction between the compound (1) and an aldehyde or ketone, which is a compound with crosslinking reactivity.

The aldehyde is not particularly limited as long as it is used when making the compound (1) novolac. The aldehyde is used alone as one kind or in combination of two or more kinds. In addition to the aldehyde, one or more ketones can also be used in combination. The aldehyde is preferably one or more selected from the group consisting of benzaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, and furfural from the viewpoint that high heat resistance can be exhibited, and it is preferably one or more selected from the group consisting of benzaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, and furfural from the viewpoint of improving etching resistance. Formaldehyde is more preferable. The amount of the aldehyde used is not particularly limited, but it is preferably 0.2 to 5 mol and more preferably 0.5 to 2 mol based on 1 mol of the compound (1).

The ketone is not particularly limited as long as it is used when making the compound (1) novolac. The ketone is used alone as one kind or in combination of two or more kinds. The ketone is preferably one or more selected from the group consisting of cyclopentanone, cyclohexanone, norbornanone, tricyclohexanone, tricyclodecanone, adamantanone, fluorenone, benzofluorenone, acenaphthenequinone, acenaphthenone, anthraquinone, acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl from the viewpoint that high heat resistance can be exhibited, and it is preferably one or more selected from the group consisting of acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl from the viewpoint of improving etching resistance. The amount of the ketone used is not particularly limited, but it is preferably 0.2 to 5 mol and more preferably 0.5 to 2 mol based on 1 mol of the compound (1).

A catalyst can also be used in the condensation reaction between the compound (1) and the aldehyde or ketone. The acid catalyst or base catalyst to be used here can be arbitrarily selected for use from publicly known catalysts and is not particularly limited. Examples of such an acid catalyst or base catalyst are the same as those described for the method for producing the compound (1). These catalysts are used alone as one kind or in combination of two or more kinds. Among these, an organic acid and a solid acid are preferable from the viewpoint of production, and hydrochloric acid or sulfuric acid is preferable from the viewpoint of production such as easy availability and handleability. The amount of the acid catalyst used can be arbitrarily set according to, for example, the kinds of the raw materials used and catalyst used, as well as the reaction conditions, and is not particularly limited, but is preferably 0.01 to 100 parts by mass based on 100 parts by mass of the reaction raw materials.

When the compound (3) is produced through a copolymerization reaction between the compound (1) and a compound having a non-conjugated double bond, such as indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, 5-vinylnorborn-2-ene, α-pinene, β-pinene, or limonene, it is not necessary to use the aldehyde or ketone.

A reaction solvent can also be used in the condensation reaction between the compound (1) and the aldehyde or ketone. The reaction solvent in this polycondensation can be arbitrarily selected for use from publicly known solvents and the examples thereof include, but are not limited to, water, methanol, ethanol, propanol, butanol, 1-methoxy-2-propanol, tetrahydrofuran, dioxane, and a mixed solvent thereof. These solvents are used alone as one kind or in combination of two or more kinds.

The amount of the solvent used can be arbitrarily set according to, for example, the kinds of the raw materials used and catalyst used, as well as the reaction conditions, and is not particularly limited, but is preferably in the range of 0 to 2000 parts by mass based on 100 parts by mass of the reaction raw materials. Furthermore, the reaction temperature can be arbitrarily selected according to the reactivity of the reaction raw materials and is not particularly limited, but is usually in the range of 10 to 200° C. Note that examples of the reaction method include a method in which the compound (1), the aldehyde and/or ketone, and the catalyst are fed in a batch, and a method in which the compound (1) and the aldehyde and/or ketone are dripped successively in the presence of the catalyst.

After the polycondensation reaction terminates, isolation of the obtained resin can be carried out according to a conventional method, and is not particularly limited. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the target compound (for example, the resin that has been made novolac) can be obtained.

The compound (3) may be a homopolymer of the compound (1), or may be a copolymer between the compound (1) and a phenol other than the compound (1). Here, examples of the copolymerizable phenol include, but are not limited to, phenol, cresol, dimethylphenol, trimethylphenol, butylphenol, phenylphenol, diphenylphenol, naphthylphenol, resorcinol, methylresorcinol, catechol, butylcatechol, methoxyphenol, methoxyphenol, propylphenol, pyrogallol, and thymol.

The compound (3) may be a copolymer with a monomer that is polymerizable with the compound (1) and is other than the phenol described above (hereinafter, also referred to as “copolymerization monomer”). Examples of such a copolymerization monomer include, but are not limited to, naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornaene, pinene, and limonene. The compound (3) may be a copolymer of two or more components (for example, a binary to quaternary system) composed of the compound (1) and the above-described phenol, may be a copolymer of two or more components (for example, a binary to quaternary system) composed of the compound (1) and the above-described copolymerization monomer, or may be a copolymer of three or more components (for example, a tertiary to quaternary system) composed of the compound (1), the above-described phenol, and the above-described copolymerization monomer.

The weight average molecular weight (Mw) of the compound (3) is not particularly limited, but it is, in terms of polystyrene through GPC measurement, preferably 300 to 100,000, more preferably 500 to 30,000, and still more preferably 750 to 20,000. In addition, the compound (3) preferably has a dispersity (weight average molecular weight Mw / number average molecular weight Mn) within the range of 1 to 7 from the viewpoint of enhancing crosslinking efficiency while suppressing volatile components during baking.

It is preferable that the compound (1) and the compound (3) each have high solubility in a solvent from the viewpoint of easier application to a wet process, etc. More specifically, in the case of using propylene glycol monomethyl ether (hereinafter, also referred to as “PGME”) and/or propylene glycol monomethyl ether acetate (hereinafter, also referred to as “PGMEA”) as a solvent, it is preferable that the compound (1) and the compound (3) each have a solubility of 10% by mass or more in the solvent. Here, the solubility in PGME and/or PGMEA is defined as “total mass of the compound (1) and the compound (3) / (total mass of the compound (1) and the compound (3) + mass of the solvent) × 100 (% by mass)”. For example, the compound (1) and the compound (3) with a total mass of 10 g are evaluated as having high solubility in 90 g of PGMEA when the solubility of the compound (1) and the compound (3) in PGMEA is “10% by mass or more”; and they are evaluated as not having high solubility when the solubility is “less than 10% by mass”. The above “total mass of the compound (1) and the compound (3)” is the mass of the compound (1) if the first composition does not contain the compound (3) but contains the compound (1). Alternatively, if the first composition does not contain compound (1) but does contain compound (3), it is the mass of the compound (3). The same applies below.

Specific examples of the compound (1) and the compound (3) include compounds represented by the following formulas. However, the compound (1) and the compound (3) are not limited to the compounds represented by the following formulas.

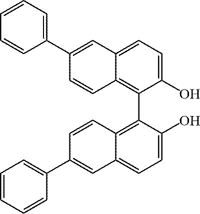 [SMILES: Oc1ccc2cc(-c3ccccc3)ccc2c1-c1c(O)ccc2cc(-c3ccccc3)ccc12]

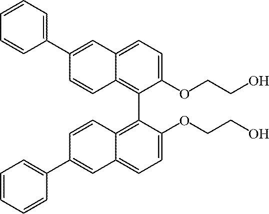 [SMILES: OCCOc1ccc2cc(-c3ccccc3)ccc2c1-c1c(OCCO)ccc2cc(-c3ccccc3)ccc12]

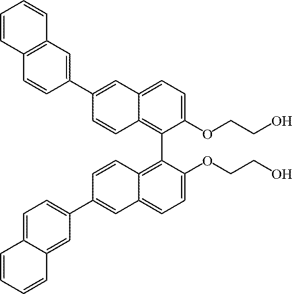 [SMILES: OCCOc1ccc2cc(-c3ccc4ccccc4c3)ccc2c1-c1c(OCCO)ccc2cc(-c3ccc4ccccc4c3)ccc12]

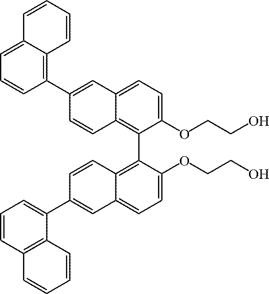 [SMILES: OCCOc1ccc2cc(-c3cccc4ccccc34)ccc2c1-c1c(OCCO)ccc2cc(-c3cccc4ccccc34)ccc12]

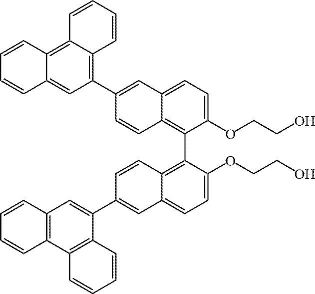 [SMILES: OCCOc1ccc2cc(-c3cc4ccccc4c4ccccc34)ccc2c1-c1c(OCCO)ccc2cc(-c3cc4ccccc4c4ccccc34)ccc12]

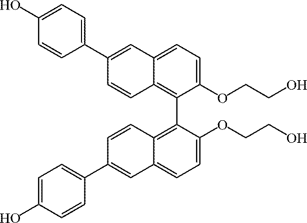 [SMILES: OCCOc1ccc2cc(-c3ccc(O)cc3)ccc2c1-c1c(OCCO)ccc2cc(-c3ccc(O)cc3)ccc12]

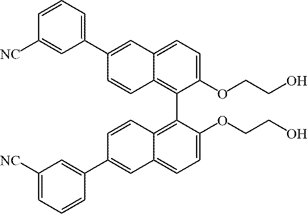

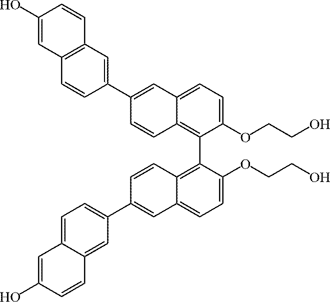 [SMILES: OCCOc1ccc2cc(-c3ccc4cc(O)ccc4c3)ccc2c1-c1c(OCCO)ccc2cc(-c3ccc4cc(O)ccc4c3)ccc12]

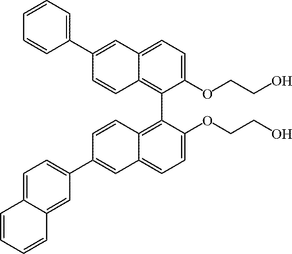 [SMILES: OCCOc1ccc2cc(-c3ccccc3)ccc2c1-c1c(OCCO)ccc2cc(-c3ccc4ccccc4c3)ccc12]

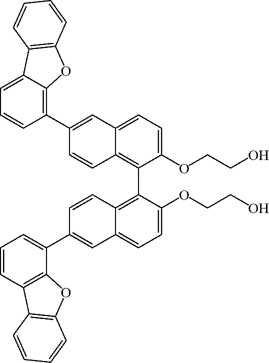 [SMILES: OCCOc1ccc2cc(-c3cccc4c3oc3ccccc34)ccc2c1-c1c(OCCO)ccc2cc(-c3cccc4c3oc3ccccc34)ccc12]

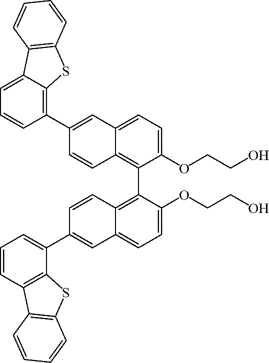 [SMILES: OCCOc1ccc2cc(-c3cccc4c3sc3ccccc34)ccc2c1-c1c(OCCO)ccc2cc(-c3cccc4c3sc3ccccc34)ccc12]

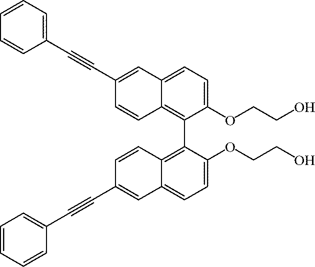 [SMILES: OCCOc1ccc2cc(C#Cc3ccccc3)ccc2c1-c1c(OCCO)ccc2cc(C#Cc3ccccc3)ccc12]

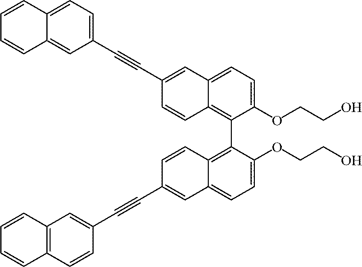 [SMILES: OCCOc1ccc2cc(C#Cc3ccc4ccccc4c3)ccc2c1-c1c(OCCO)ccc2cc(C#Cc3ccc4ccccc4c3)ccc12]

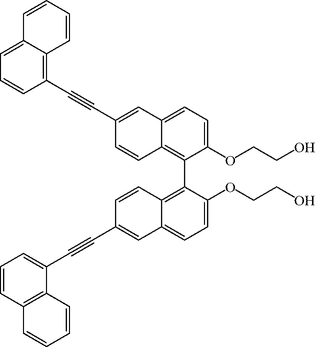 [SMILES: OCCOc1ccc2cc(C#Cc3cccc4ccccc34)ccc2c1-c1c(OCCO)ccc2cc(C#Cc3cccc4ccccc34)ccc12]

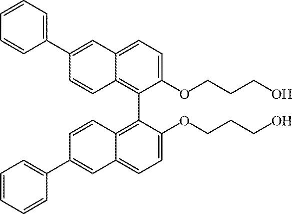 [SMILES: OCCCOc1ccc2cc(-c3ccccc3)ccc2c1-c1c(OCCCO)ccc2cc(-c3ccccc3)ccc12]

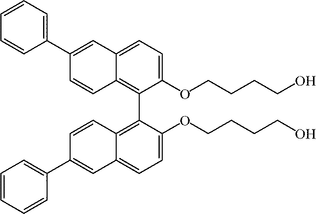 [SMILES: OCCCCOc1ccc2cc(-c3ccccc3)ccc2c1-c1c(OCCCCO)ccc2cc(-c3ccccc3)ccc12]

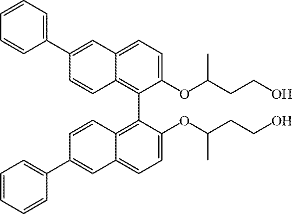 [SMILES: CC(CCO)Oc1ccc2cc(-c3ccccc3)ccc2c1-c1c(OC(C)CCO)ccc2cc(-c3ccccc3)ccc12]

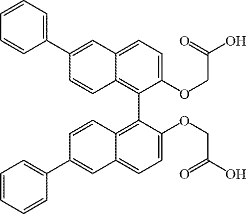 [SMILES: O=C(O)COc1ccc2cc(-c3ccccc3)ccc2c1-c1c(OCC(=O)O)ccc2cc(-c3ccccc3)ccc12]

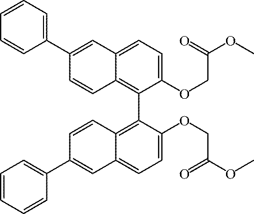 [SMILES: COC(=O)COc1ccc2cc(-c3ccccc3)ccc2c1-c1c(OCC(=O)OC)ccc2cc(-c3ccccc3)ccc12]

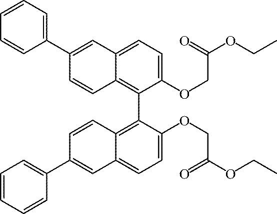 [SMILES: CCOC(=O)COc1ccc2cc(-c3ccccc3)ccc2c1-c1c(OCC(=O)OCC)ccc2cc(-c3ccccc3)ccc12]

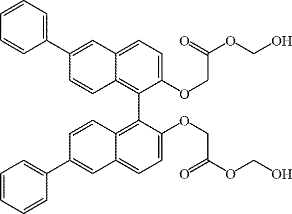 [SMILES: O=C(COc1ccc2cc(-c3ccccc3)ccc2c1-c1c(OCC(=O)OCO)ccc2cc(-c3ccccc3)ccc12)OCO]

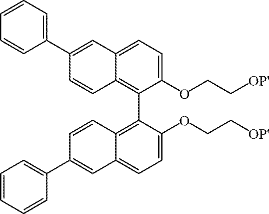

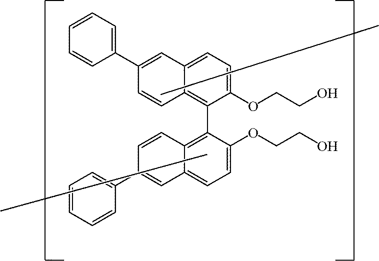

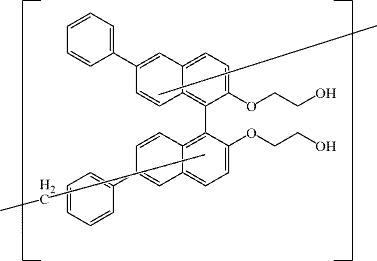

In the formula, OP’ each independently represents a crosslinkable group or a dissociable group.

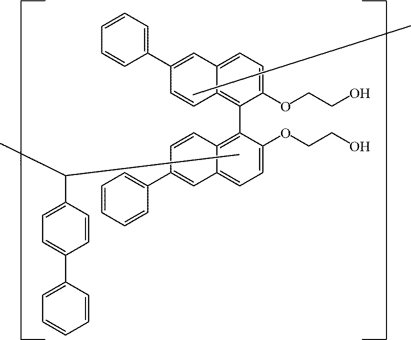

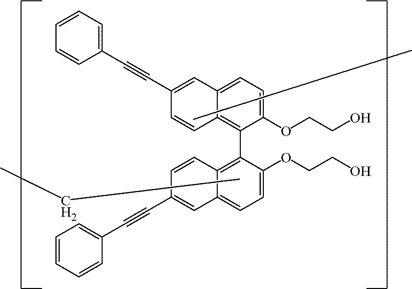

### Second Film Forming Composition for Lithography

The second film forming composition for lithography of the present embodiment (hereinafter, also simply referred to as “second composition”) contains at least one selected from: a compound represented by formula (4) (hereinafter, also referred to as “compound (4)”); a compound represented by formula (5) (hereinafter, also referred to as “compound (5)”); and a resin obtained using them as a monomer (hereinafter, also referred to as “resin”). Also, it is preferable that the compound represented by formula (4) be a compound represented by formula (6) (hereinafter, also referred to as “compound (6)”) and the compound represented by formula (5) be a compound represented by formula (7) (hereinafter, also referred to as “compound (7)”). In the present embodiment, the compound represented by formula (4); the compound represented by formula (5); the compound represented by formula (6); the compound represented by formula (7); and the resin obtained using them as a monomer, are also referred to as “the compound (4) to the compound (7) and the resin”.

According to the second composition, it is possible to provide a composition that is useful as a film forming material for lithography, having high solubility in organic solvents, excellent storage stability and thin film formability, high etching resistance, high sensitivity, and excellent resist pattern formability, and satisfying a good balance of these physical properties at high levels. Also, by using this composition, it is possible to provide a resist pattern formation method and a circuit pattern formation method.

The compound (4) to the compound (7) and the resin of the present embodiment have a plurality of polar groups, and thus have excellent solubility in organic solvents. In addition, the compound (4) to the compound (7) and the resin have a relatively high content rate of aromatic rings and excellent crosslinking reactivity, which also results in excellent heat resistance.

The second composition containing the compound (4) to the compound (7) and the resin of the present embodiment also has excellent solubility in organic solvents, is applicable to a wet process, and has excellent storage stability, thin film formability, smoothing properties, and heat resistance. Moreover, in the second composition, film deterioration during high temperature baking is suppressed, and a film for lithography excellent in etching resistance against oxygen plasma etching or the like can be formed. Furthermore, since the second composition has high sensitivity and is also excellent in adhesiveness to a resist film when used as an underlayer film, use of the second composition can form an excellent resist pattern. Therefore, the second composition is suitably used as a material for film formation for lithography.

### Compound

A compound contained in the second film forming composition for lithography is the compound represented by formula (4) and/or the compound represented by formula (5).

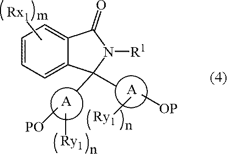

In formula (4),
A each independently represents an aromatic group having 6 to 10 carbon atoms,
P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group,
R¹ represents an aromatic substituent having 6 to 10 carbon atoms or an alkyl group having 1 to 20 carbon atoms,
Rx₁ each independently represents a hydrogen atom, an alkyl group having 1 to 20 carbon atoms, an alkoxy group having 1 to 20 carbon atoms, or a halogen,
Ry₁ each independently represents a hydrogen atom, an alkyl group having 1 to 20 carbon atoms, an alkoxy group having 1 to 20 carbon atoms, or a halogen, and
m represents an integer of 0 to 4 and n represents an integer of 0 to 4.

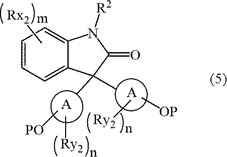

In formula (5),
A each independently represents an aromatic group having 6 to 10 carbon atoms,
P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group,
R² represents an aromatic substituent having 6 to 10 carbon atoms or an alkyl group having 1 to 20 carbon atoms,
Rx₂ each independently represents a hydrogen atom, an alkyl group having 1 to 20 carbon atoms, an alkoxy group having 1 to 20 carbon atoms, or a halogen,
Ry₂ each independently represents a hydrogen atom, an alkyl group having 1 to 20 carbon atoms, an alkoxy group having 1 to 20 carbon atoms, or a halogen, and
m represents an integer of 0 to 4 and n represents an integer of 0 to 4.

It is preferable that the compound (4) and the compound (5) be the compound represented by formula (6) and the compound represented by formula (7), respectively, since a good balance of solubility in organic solvents, storage stability, thin film formability, heat resistance, etching resistance, sensitivity, and resist pattern formability can be simultaneously satisfied at high levels as a film forming material for lithography.

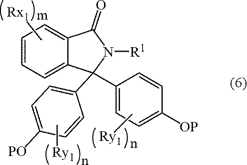

In formula (6), P, R′, Rx₁ Ry₁, m, and n are the same as defined in formula (4).

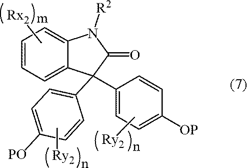

In formula (7), P, R², Rx₂ Ry₂, m, and n are the same as defined in formula (5).

A each independently represents an aromatic group having 6 to 10 carbon atoms, and since excellent etching resistance is obtained, it is preferably a phenyl group or a naphthyl group, and more preferably a phenyl group.

P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group, and from the viewpoint of thin film formability and suitably suppressed film deterioration during high temperature baking, it is preferably a hydrogen atom, a crosslinkable group, or a dissociable group, and more preferably a hydrogen atom.

The alkyl group having 1 to 30 carbon atoms may be linear, branched, or cyclic, and examples thereof include a methyl group, a tert-butyl group, a cyclohexyl group, and an adamantyl group.

Examples of the aryl group having 6 to 30 carbon atoms include a phenyl group, a tolyl group, and a naphthyl group. Note that the aryl group may further have a substituent such as a halogen atom and an alkyl group having 1 to 5 carbon atoms.

Examples of the alkenyl group having 2 to 20 carbon atoms include a vinyl group, an allyl group, a 4-pentenyl group, an isopropenyl group, an isopentenyl group, a 2-heptenyl group, a 2-octenyl group, and a 2-nonenyl group.

Examples of the alkynyl group having 2 to 20 carbon atoms include an ethynyl group and a propargyl group.

The “crosslinkable group” in the present embodiment refers to a group that crosslinks in the presence of a catalyst or without a catalyst. Examples of such a crosslinkable group include an alkoxy group having 1 to 20 carbon atoms, a group having an allyl group, a group having a (meth)acryloyl group, a group having an epoxy (meth)acryloyl group, a group having a hydroxyl group, a group having a urethane (meth)acryloyl group, a group having a glycidyl group, a group having a vinyl-containing phenylmethyl group, a group having a group having various alkynyl groups, a group having a carbon-carbon double bond, a group having a carbon-carbon triple bond, and a group that crosslinks in the presence of a catalyst or without a catalyst among a group containing these groups and others. The “group containing these groups” described above is preferably, for example, an alkoxy group represented by —ORx (Rx is a group having an allyl group, a group having a (meth)acryloyl group, a group having an epoxy (meth)acryloyl group, a group having a hydroxyl group, a group having a urethane (meth)acryloyl group, a group having a glycidyl group, a group having a vinyl-containing phenylmethyl group, a group having a group having various alkynyl groups, a group having a carbon-carbon double bond, a group having a carbon-carbon triple bond, and a group containing these groups). Note that, in the present embodiment, for each functional group (excluding the crosslinkable group) described above as constituting the compounds, if there is an overlap with the crosslinkable group, based on the presence or absence of crosslinkability, one without crosslinkability is treated as corresponding to the functional group, and one with crosslinkability is treated as corresponding to the crosslinkable group.

Examples of the alkoxy group having 1 to 20 carbon atoms include a methoxy group, an ethoxy group, a propoxy group, an isopropoxy group, a n-butoxy group, an isobutoxy group, a tert-butoxy group, a n-hexanoxy group, and a 2-methylpropoxy group.

Examples of the group having an allyl group include groups represented by formulas (X-1a) and (X-1b).

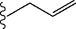

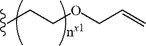

In formula (X-1b), n^{X1} is an integer of 1 to 5.

Examples of the group having a (meth)acryloyl group include groups represented by formulas (X-2a) to (X-2c).

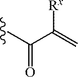

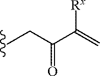

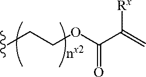

In formula (X-2c), n^{x2} is an integer of 1 to 5, and in formulas (X-2a) to (X-2c), R^{X} is a hydrogen atom or a methyl group.

Examples of the group having an epoxy (meth)acryloyl group include a group represented by the following formula (X-3). The epoxy (meth)acryloyl group refers to a group generated through a reaction between an epoxy (meth)acrylate and a hydroxyl group.

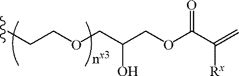

In formula (X-3), n^{x3} is an integer of 0 to 5, and since excellent heat resistance and etching resistance are obtained, it is preferably 0. R^{X} is a hydrogen atom or a methyl group, and since excellent curability is obtained, it is preferably a methyl group.

Examples of the group having a urethane (meth)acryloyl group include a group represented by formula (X-4).

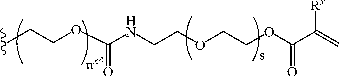

In formula (X-4), n^{x4} is an integer of 0 to 5, and since excellent heat resistance and etching resistance are obtained, it is preferably 0. s is an integer of 0 to 3, and since excellent heat resistance and etching resistance are obtained, it is preferably 0. R^{X} is a hydrogen atom or a methyl group, and because excellent curability is obtained, it is preferably a methyl group.

Examples of the group having a hydroxyl group include groups represented by the following formulas (X-5a) to (X-5e).

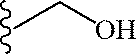

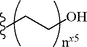

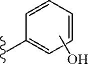

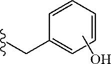

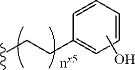

In formulas (X-5b) and (X-5e), n^{x5} is an integer of 1 to 5, and since excellent heat resistance and etching resistance are obtained, it is preferably 1.

Examples of the group having a glycidyl group include groups represented by formulas (X-6a) to (X-6c).

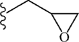

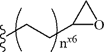

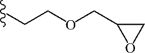

In formula (X-6b), n^{x6} is an integer of 1 to 5.

Examples of the group having a vinyl-containing phenylmethyl group include groups represented by formulas (X-7a) and (X-7b).

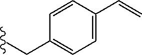

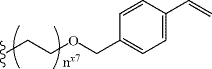

In formula (X-7b), n^{x7} is an integer of 1 to 5, and since excellent heat resistance and etching resistance are obtained, it is preferably 1.

Examples of the group having various alkynyl groups include groups represented by the following formulas (X-8a) to (X-8h).

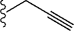

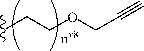

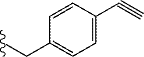

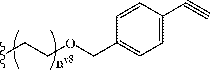

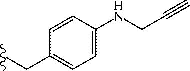

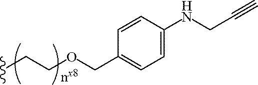

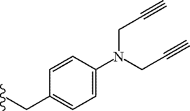

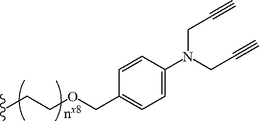

In formulas (X-8b), (X-8d), (X-8f), and (X-8h), n^{x8} is an integer of 1 to 5.

Examples of the carbon-carbon double bond-containing group include a (meth)acryloyl group, a substituted or unsubstituted vinylphenyl group, and a group represented by formula (X-9).

In addition, examples of the carbon-carbon triple bond-containing group include a substituted or unsubstituted ethynyl group, a substituted or unsubstituted propargyl group, and groups represented by formulas (X-10a) and (X-10b).

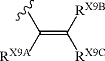

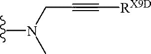

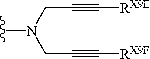

In formula (X-9), R^{X9A}, R^{X9B} and R^{X9C} are each independently a hydrogen atom or a monovalent hydrocarbon group having 1 to 20 carbon atoms. In formulas (X-10a) and (X-10b), R^{X9D}, R^{X9E} and R^{X9F} are each independently a hydrogen atom or a monovalent hydrocarbon group having 1 to 20 carbon atoms.

In the present embodiment, the “dissociable group” refers to a group that is dissociated in the presence of a catalyst or without a catalyst. Among dissociable groups, an acid dissociable group refers to a group that is cleaved in the presence of an acid to cause a change into an alkali soluble group or the like.

Examples of the alkali soluble group include a phenolic hydroxyl group, a carboxyl group, a sulfonic acid group, and a hexafluoroisopropanol group. Among these, from the viewpoint of the easy availability of an introduction reagent, a phenolic hydroxyl group and a carboxyl group are preferable, and a phenolic hydroxyl group is more preferable.

The acid dissociable group preferably has the property of causing chained cleavage reaction in the presence of an acid for achieving pattern formation with high sensitivity and high resolution.

The acid dissociable group can be arbitrarily selected for use from among, for example, those proposed in hydroxystyrene resins, (meth)acrylic acid resins, and the like for use in chemically amplified resist compositions for KrF or ArF.

Examples of the acid dissociable group can include those described in International Publication No. WO 2016/158168. Examples of the acid dissociable group also include a 1-substituted ethyl group, a 1-substituted n-propyl group, a 1-branched alkyl group, a silyl group, an acyl group, a 1-substituted alkoxymethyl group, a cyclic ether group, an alkoxycarbonyl group (such as —C(O)OC(CH₃)₃), and an alkoxycarbonylalkyl group (such as —(CH₂)ₙC(O)OC(CH₃)₃, wherein n = 1 to 4), which have the property of being dissociated by an acid. Note that, in the present embodiment, for each functional group (excluding the dissociable group) described above as constituting the compounds, if there is an overlap with the dissociable group, based on the presence or absence of dissociability, one without dissociability is treated as corresponding to the functional group, and one with dissociability is treated as corresponding to the dissociable group.

Examples of the substituent for the dissociable group include a halogen atom, an alkyl group, an aryl group, an aralkyl group, an alkynyl group, an alkenyl group, an acyl group, an alkoxycarbonyl group, an alkyloyloxy group, an aryloyloxy group, a cyano group, and a nitro group. These groups may have a heteroatom.

Examples of the halogen atom include a fluorine atom, a chlorine atom, a bromine atom, and an iodine atom.

As the alkyl group, those described above can be referred to, and examples thereof include an alkyl group having 1 to 10 carbon atoms such as a methyl group, a tert-butyl group, a cyclohexyl group, and an adamantyl group.

As the aryl group, those described above can be referred to, but an aryl group having 6 to 20 carbon atoms is preferable. Note that the aryl group may further have a substituent such as a halogen atom or an alkyl group having 1 to 5 carbon atoms.

Examples of the aralkyl group include a benzyl group and a phenethyl group. Note that the aralkyl group may further have a substituent such as a halogen atom and an alkyl group having 1 to 5 carbon atoms.

As the alkynyl group, those described above can be referred to.

Examples of the acyl group include an aliphatic acyl group having 1 to 6 carbon atoms such as a formyl group and an acetyl group, and an aromatic acyl group such as a benzoyl group.

Examples of the alkoxycarbonyl group include an alkoxycarbonyl group having 2 to 5 carbon atoms such as a methoxycarbonyl group.

Examples of the alkyloyloxy group include an acetoxy group.

Examples of the aryloxy group include a benzoyloxy group.

Examples of the heteroatom include an oxygen atom, a sulfur atom, a selenium atom, a nitrogen atom, and a phosphorus atom.

A carbon atom of each group may be substituted with the heteroatom.

Note that, in the case of including the substituents, the number of carbon atoms in each group described in the present embodiment is the total number of carbon atoms including the substituents.

R¹ represents an aromatic substituent having 6 to 10 carbon atoms or an alkyl group having 1 to 20 carbon atoms, but among these, it is preferably an aromatic substituent having 6 to 10 carbon atoms or an alkyl group having 1 to 10 carbon atoms, and it is more preferably a phenyl group. Examples of such a group include a phenyl group, a methyl group, an ethyl group, a n-propyl group, and an i-propyl group.

Rx₁ represents a hydrogen atom, an alkyl group having 1 to 20 carbon atoms, an alkoxy group having 1 to 20 carbon atoms, or a halogen, but among these, it is preferably a hydrogen atom or an alkyl group having 1 to 10 carbon atoms, it is more preferably a hydrogen atom or an alkyl group having 1 to 3 carbon atoms, and it is still more preferably a hydrogen atom. Examples of such a group include a hydrogen atom, a methyl group, an ethyl group, a n-propyl group, and an i-propyl group. m represents an integer of 0 to 4.

Ry₁ represents a hydrogen atom, an alkyl group having 1 to 20 carbon atoms, an alkoxy group having 1 to 20 carbon atoms, or a halogen, but among these, it is preferably a hydrogen atom or an alkyl group having 1 to 10 carbon atoms, it is more preferably a hydrogen atom or an alkyl group having 1 to 3 carbon atoms, and it is still more preferably a hydrogen atom. Examples of such a group include a hydrogen atom, a methyl group, an ethyl group, a n-propyl group and an i-propyl group. n represents an integer of 0 to 4.

R² represents an aromatic substituent having 6 to 10 carbon atoms or an alkyl group having 1 to 20 carbon atoms, but among these, it is preferably an aromatic substituent having 6 to 10 carbon atoms or an alkyl group having 1 to 10 carbon atoms, it is more preferably an alkyl group having 1 to 3 carbon atoms, and it is still more preferably a methyl group. Examples of such a group include a phenyl group, a naphthyl group, a methyl group, an ethyl group, a n-propyl group, and an i-propyl group.

Rx₂ represents a hydrogen atom, an alkyl group having 1 to 20 carbon atoms, an alkoxy group, or a halogen, but among these, it is preferably a hydrogen atom or an alkyl group having 1 to 10 carbon atoms, it is more preferably a hydrogen atom or an alkyl group having 1 to 3 carbon atoms, and it is still more preferably a hydrogen atom. Examples of such a group include a hydrogen atom, a methyl group, an ethyl group, a n-propyl group, and an i-propyl group. m represents an integer of 0 to 4.

Ry₂ represents a hydrogen atom, an alkyl group having 1 to 20 carbon atoms, an alkoxy group, or a halogen, but among these, it is preferably a hydrogen atom or an alkyl group having 1 to 10 carbon atoms, it is more preferably a hydrogen atom or an alkyl group having 1 to 3 carbon atoms, and it is still more preferably a hydrogen atom. Examples of such a group include a hydrogen atom, a methyl group, an ethyl group, a n-propyl group and an i-propyl group. n represents an integer of 0 to 4.

### Methods for Producing Compounds

Examples of the method for producing the compound (4) include, but are not limited to, the method produced from a phenol represented by formula (4-1) according to a method known as a dehydration reaction.

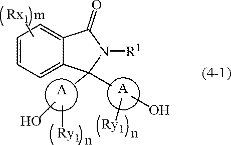

In formula (4-1), A, R¹, Rx₁ Ry₁, m, and n are the same as defined in formula (4).

The phenol represented by formula (4-1) can be obtained through a dehydration reaction between a phenolphthalein derivative represented by formula (4-2) and an amine derivative or an aniline derivative, according to a publicly known and established method. As such a dehydration reaction, for example, Japanese Patent Laid-Open No. 2005-290378 can be referred to. It may also be synthesized by other publicly known methods.

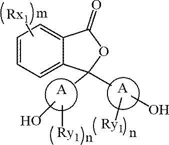

In formula (4-2), A, R¹, Rx₁ Ry₁, m, and n are the same as defined in formula (4).

Examples of the amine derivative include methylamine, ethylamine, propylamine, isopropylamine, butylamine, sec-butylamine, tert-butylamine, amylamine, hexylamine, heptylamine, octylamine, nonylamine, decylamine, and cyclohexylamine.

Examples of the aniline derivative include aniline, o-methylaniline, m-methylaniline, p-methylaniline, o-methoxyaniline, m-methoxyaniline, p-methoxyaniline, o-ethylaniline, m-ethylaniline, p-ethylaniline, 2,3-dimethylaniline, 2,4-dimethylaniline, 2,5-dimethylaniline, 3,4-dimethylaniline, 3,5-dimethylaniline, o-chloromethylaniline, m-chloromethylaniline, p-chloromethylaniline, o-trifluoromethylaniline, m-trifluoromethylaniline, p-trifluoromethylaniline, o-chloroaniline, m-chloroaniline, p-chloroaniline, o-fluoroaniline, m-fluoroaniline, p-fluoroaniline, o-bromoaniline, m-bromoaniline, p-bromoaniline, o-carbomethoxyaniline, m-carbomethoxyaniline, p-carbomethoxyaniline, o-acetoxyaniline, m-acetoxyaniline, p-acetoxyaniline, 1-naphthylamine, 2-naphthylamine, 2-aminophenol, 3-aminophenol, 4-aminophenol, 4-amino-2-fluorophenol, 4-amino-2-chlorophenol, 4-amino-3-chlorophenol, 1-amino-2-naphthol, 2-amino-1-naphthol, 3-amino-2-naphthol, 4-amino-1-naphthol, 5-amino-1-naphthol, 5-amino-2-naphthol, 6-amino-1-naphthol, 8-amino-2-naphthol, 2-amino-m-cresol, 2-amino-p-cresol, 3-amino-o-cresol, 3-amino-p-cresol, 4-amino-m-cresol, 4-amino-o-cresol, 5-amino-o-cresol, 6-amino-m-cresol, 4-amino-3,5-xylenol, and 3-hydroxy-4-methoxyaniline.

As for the method for producing the compound (5), it can be produced, for example, from a phenol represented by formula (5-1) according to a method known as a dehydration reaction.

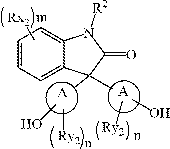

In formula (5-1), A, R², Rx₂ Ry₂, m, and n are the same as defined in formula (5).

As the method for producing the phenol represented by formula (5-1), a publicly known method can be utilized, and examples thereof include, but are not limited to, the method synthesized from an indoline-2,3-dione represented by formula (5-2) and a phenol. As such a synthesis method, for example, Japanese Patent Laid-Open No. 2002-179649 can be referred to.

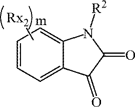

In formula (5-2), R² and Rx² are the same as defined in formula (5).

In the method for producing the phenol represented by formula (5-1), examples of the phenol that is allowed to react with the indoline-2,3-dione represented by formula (5-2) include phenol, o-cresol, m-cresol, p-cresol, o-fluorophenol, m-fluorophenol, p-fluorophenol, o-chlorophenol, m-chlorophenol, p-chlorophenol, o-bromophenol, m-bromophenol, p-bromophenol, p-tert-butylphenol, p-nonylphenol, 2,4-xylenol, 2,5-xylenol, 3,4-xylenol, 3,5-xylenol, and resorcinol.

As the method for producing the compound represented by formula (5-2), a publicly known method can be utilized.

### Resin

The second film forming composition for lithography can contain a resin obtained by polymerizing any one or more of the compound (4) to the compound (7) as monomers. The resin may be a homopolymer comprising one kind of monomer or a copolymer combining two or more kinds of monomers as appropriate. The copolymer may be a block copolymer or a random copolymer. The resin may be an oligomeric product or a polymeric product. In addition, it is preferable that the resin of the present embodiment be a resin obtained by using any one or more compounds among the compound (4) to the compound (7) as monomers and allowing these monomers to react with a monomer with crosslinking reactivity. Such a resin may be an oligomerized resin or a polymeric product. Examples of such a resin include a resin represented by formula (8) (hereinafter, also referred to as “resin (8)”. When used as a material for underlayer film formation for lithography, it is preferable that the second composition contain the resin represented by formula (8).

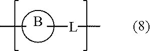

In formula (8), B is any one or more selected from: a constituent unit derived from the compound represented by formula (4); a constituent unit derived from the compound represented by formula (5); a constituent unit derived from the compound represented by formula (6); and a constituent unit derived from the compound represented by formula (7). When the resin has a plurality of these constituent units, it may be in a block form or in a random form. It is preferable that the resin be obtained by allowing any one selected from: a constituent unit derived from the compound represented by formula (4); a constituent unit derived from the compound represented by formula (5); a constituent unit derived from the compound represented by formula (6); and a constituent unit derived from the compound represented by formula (7) to react with a monomer with crosslinking reactivity.

L is a single bond, a linear or branched alkylene group having 1 to 20 carbon atoms and optionally having a substituent, or an arylene group having 1 to 20 carbon atoms and optionally having a substituent.

When there are a plurality of B and/or L, they are independent of each other.

The substituted group means a group in which one or more hydrogen atoms in a functional group have been replaced by an atom other than hydrogen atom or a functional substituent, unless otherwise defined. The number of substituents is not limited, and may be one or more.

Examples of the linear or branched alkylene group having 1 to 20 carbon atoms include a methylene group, an ethylene group, a propylene group, a 2,2-dimethylpropylene group, a butylene group, a pentylene group, a hexylene group, a heptylene group, an octylene group, a nonylene group, a decylene group, a dodecylene group, an undecylene group, a tridecylene group, a tetradecylene group, a pentadecylene group, a hexadecylene group, a neopentylene group, a dimethylbutylene group, a methylhexylene group, an ethylhexylene group, a dimethylhexylene group, a trimethylhexylene group, a methylheptylene group, a dimethylheptylene group, a trimethylheptylene group, a tetramethylheptylene group, an ethylheptylene group, a methyloctylene group, a methylnonylene group, a methyldecylene group, a methyldodecylene group, a methylundecylene group, a methyltridecylene group, a methyltetradecylene group, and a methylpentadecylene group.

Examples of the arylene group having 1 to 20 carbon atoms include a phenylene group such as a 1,4-phenylene group, a 1,3-phenylene group, and a 1,2-phenylene group; a naphthalenediyl group such as a 1,4-naphthalenediyl group, a 1,5-naphthalenediyl group, a 2,6-naphthalenediyl group, and a 2,7-naphthalenediyl group; an anthracenediyl group such as a 1,4-anthracenediyl group, a 1,5-anthracenediyl group, a 2,6-anthracenediyl group, and a 9,10-anthracenediyl group; a phenanthrenediyl group such as a 2,7-phenanthrenediyl group; a dihydrophenanthrenediyl group such as a 9,10-dihydrophenanthrene-2,7-diyl group; a naphthacenediyl group such as a 1,7-naphthacenediyl group, a 2,8-naphthacenediyl group, and a 5,12-naphthacenediyl group; a fluorenediyl group such as a 2,7-fluorenediyl group and a 3,6-fluorenediyl group; a pyrenediyl group such as a 1,6-pyrenediyl group, a 1,8-pyrenediyl group, a 2,7-pyrenediyl group, and a 4,9-pyrenediyl group; a perylenediyl group such as a 3,8-perylenediyl group, a 3,9-perylenediyl group, and a 3,10-perylenediyl group; and a spirofluorenediyl group such as a 9,9′-spirofluorene-2,7-diyl group, a 9,9′-spirofluorene-3,6-diyl group, and a 9,9′-spirofluorene-2,2′-diyl group.

The compound with crosslinking reactivity may be any compound as long as it can oligomerize or polymerize the compound (4) to the compound (7), and examples thereof include an aldehyde, a ketone, a carboxylic acid, a halogen-containing compound such as an acid halide and an alkyl halide, an amino compound, an imino compound, an isocyanate compound, and an unsaturated hydrocarbon group-containing compound. These compounds with crosslinking reactivity can be used alone as one kind or in combination of two or more kinds.

These compounds with crosslinking reactivity can suitably crosslink the aromatic groups of the compound (4) to the compound (7) in the presence of a catalyst to form a more stable film. Therefore, by using a resin crosslinked with these compounds, it is possible to obtain a composition that is useful as a film forming material for lithography, having high solubility in organic solvents, excellent storage stability and thin film formability, high etching resistance, excellent heat resistance, high sensitivity, and excellent resist pattern formability, and satisfying a good balance of these physical properties at high levels.

As the resin (8), a resin that has been made novolac, obtained through a condensation reaction between the compound (4) to the compound (7) and an aldehyde or ketone, which is a compound with crosslinking reactivity, is preferable since it has excellent reactivity.

The aldehyde can be used alone as one kind or in combination of two or more kinds. In addition to the aldehyde, one or more ketones can also be used in combination.

As the aldehyde, from the viewpoint that etching resistance can be improved and high heat resistance can be exhibited, formaldehyde, benzaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, and furfural are preferable. From the point that reactivity is high, etching resistance can be improved more, and higher heat resistance can be exhibited, formaldehyde, benzaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, and furfural are more preferable. It is still more preferable to use formaldehyde. The amount of the aldehyde used is not particularly limited, but it is preferably 0.2 to 10 mol and more preferably 0.5 to 8 mol based on 1 mol of the total amount of the compound (4) to the compound (7).

As the ketone, from the viewpoint that etching resistance can be improved and high heat resistance can be exhibited, cyclopentanone, cyclohexanone, norbornanone, tricyclohexanone, tricyclodecanone, adamantanone, fluorenone, benzofluorenone, acenaphthenequinone, acenaphthenone, anthraquinone, acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl are preferable. From the point that etching resistance can be improved more and higher heat resistance can be exhibited, acetophenone, diacetylbenzene, triacetylbenzene, acetonaphthone, diphenylcarbonylnaphthalene, phenylcarbonylbiphenyl, diphenylcarbonylbiphenyl, benzophenone, diphenylcarbonylbenzene, triphenylcarbonylbenzene, benzonaphthone, diphenylcarbonylnaphthalene, phenylcarbonylbiphenyl, and diphenylcarbonylbiphenyl are more preferable. The amount of the ketone used is not particularly limited, but it is preferably 0.2 to 5 mol and more preferably 0.5 to 2 mol based on 1 mol of the total amount of the compound (4) to the compound (7).

Examples of the carboxylic acid include oxalic acid, maleic acid, fumaric acid, phthalic acid, isophthalic acid, adipic acid, and cyclohexanedicarboxylic acid.

Examples of the halogen-containing compound include a compound containing an alkyl halide group and an aryl halide group. Examples of the alkyl halide group include a trifluoromethyl group, a 1,1,1-trifluoroethyl group, a 1,1,1-trichloroethyl group, and a nonafluorobutyl group. Examples of the aryl halide group include a fluorophenyl group, a chlorophenyl group, and a 1,2,3,4,5-pentafluorophenyl group.

Examples of the amino compound include those described in International Publication No. WO 2018-016614.

Examples of the imino compound include 2,2′-iminodiethanol, ethyleneimine, propyleneimine, butyleneimine, dimethylethyleneimine, pentyleneimine, hexyleneimine, heptyleneimine, and octyleneimine.

Examples of the isocyanate compound include those described in International Publication No. WO 2018-016614.

Examples of the unsaturated hydrocarbon group-containing compound include a compound having an allyl group and a compound having an alkynyl group.

Also, by a method in which one-electron oxidation polymerization is carried out in the presence of an oxidizing agent, for example, the aromatic rings of any one or more of the compound (4) to the compound (7) only may be subjected to a polymerization reaction by themselves, in which case L in formula (8) represents a single bond.

As the oxidizing agent, a metal salt or metal complex containing copper, manganese, iron, cobalt, ruthenium, chromium, palladium, or the like, a peroxide such as hydrogen peroxide and a perchlorate, an organic peroxide, and the like can be used. Among these, the metal salt or metal complex containing copper, manganese, iron, or cobalt is preferable. The metal such as copper, manganese, iron, cobalt, ruthenium, chromium, or palladium can also be used as the oxidizing agent by reduction in the reaction system. These are included in the metal salt. The oxidizing agent may be used alone as one kind, or two or more kinds may be mixed as appropriate for use.

The resin of the present embodiment may be a homopolymer of any one or more selected from the compound (4) to the compound (7), and a resin thereof crosslinked with a compound with crosslinking reactivity, but it may also be a copolymer in which such a resin is polymerized with an additional phenol. Examples of the copolymerizable phenol include phenol, cresol, dimethylphenol, trimethylphenol, butylphenol, phenylphenol, diphenylphenol, naphthylphenol, resorcinol, methylresorcinol, catechol, butylcatechol, methoxyphenol, methoxyphenol, propylphenol, pyrogallol, and thymol.

In addition, the resin of the present embodiment may be a copolymer with a polymerizable monomer other than the additional phenol described above. Examples of the copolymerizable monomer include naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornene, pinene, and limonene. The resin of the present embodiment may be a copolymer of two or more components (for example, a binary to quaternary system) composed of the compound (4) to the compound (7) or the resin thereof crosslinked with a compound with crosslinking reactivity, and the phenol, may be a copolymer of two or more components (for example, a binary to quaternary system) composed of the compound (4) to the compound (7) or the resin thereof crosslinked with a compound with crosslinking reactivity, and the copolymerizable monomer, or may be a copolymer of three or more components (for example, a tertiary to quaternary system) composed of the compound (4) to the compound (7) or the resin thereof crosslinked with a compound with crosslinking reactivity, the phenol, and the copolymerizable monomer.

The mass average molecular weight (Mw) of the resin of the present embodiment is, for example, preferably 300 to 100,000, more preferably 500 to 30,000, and still more preferably 750 to 20,000, in terms of polystyrene through GPC measurement. In addition, the resin of the present embodiment preferably has a dispersity (mass average molecular weight Mw / number average molecular weight Mn) within the range of 1 to 7 from the viewpoint of enhancing crosslinking efficiency while suppressing volatile components during baking.

It is preferable that the compound (4) to the compound (7) and the resin have high solubility in a solvent from the viewpoint of easier application to a wet process, etc. For example, in the case of using propylene glycol monomethyl ether (hereinafter, also referred to as “PGME”), propylene glycol monomethyl ether acetate (hereinafter, also referred to as “PGMEA”), and/or cyclohexanone (hereinafter, also referred to as “CHN”) as a solvent, it is preferable that the compound (4) to the compound (7) and the resin have a solubility of 5% by mass or more in the solvent. Here, the solubility in PGME and/or PGMEA is defined as “total amount of the compound (4) to the compound (7) and the resin / (total amount of the compound (4) to the compound (7) and the resin + total amount of the solvent) × 100 (% by mass)”. For example, the compound (4) to the compound (7) and the resin with a total amount of 5 g are evaluated as having high solubility in 95 g of PGMEA when the solubility of the compound (4) to the compound (7) and the resin in PGMEA is “5% by mass or more”; and they are evaluated as not having high solubility when the solubility is “less than 5% by mass”.

Examples of the compound (4) to the compound (7) and the resin include compounds represented by the following formulas. Note that a partial structure is shown for the resin.

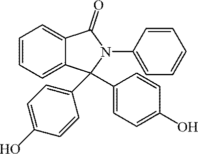 [SMILES: O=C1c2ccccc2C(c2ccc(O)cc2)(c2ccc(O)cc2)N1c1ccccc1]

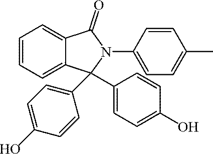 [SMILES: Cc1ccc(N2C(=O)c3ccccc3C2(c2ccc(O)cc2)c2ccc(O)cc2)cc1]

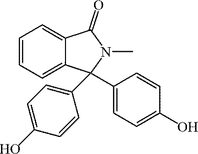 [SMILES: CN1C(=O)c2ccccc2C1(c1ccc(O)cc1)c1ccc(O)cc1]

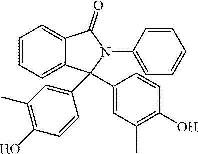 [SMILES: Cc1cc(C2(c3ccc(O)c(C)c3)c3ccccc3C(=O)N2c2ccccc2)ccc1O]

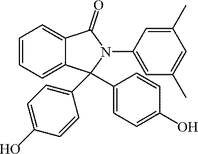 [SMILES: Cc1cc(C)cc(N2C(=O)c3ccccc3C2(c2ccc(O)cc2)c2ccc(O)cc2)c1]

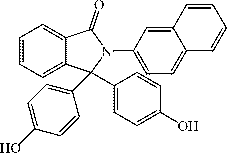 [SMILES: O=C1c2ccccc2C(c2ccc(O)cc2)(c2ccc(O)cc2)N1c1ccc2ccccc2c1]

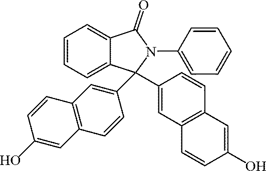 [SMILES: O=C1c2ccccc2C(c2ccc3cc(O)ccc3c2)(c2ccc3cc(O)ccc3c2)N1c1ccccc1]

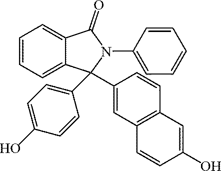 [SMILES: O=C1c2ccccc2C(c2ccc(O)cc2)(c2ccc3cc(O)ccc3c2)N1c1ccccc1]

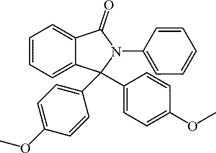

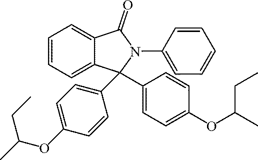 [SMILES: CCC(C)Oc1ccc(C2(c3ccc(OC(C)CC)cc3)c3ccccc3C(=O)N2c2ccccc2)cc1]

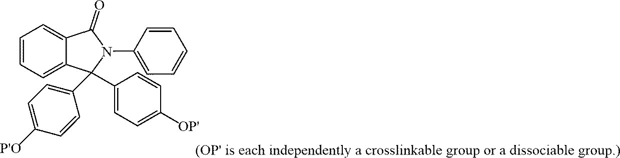

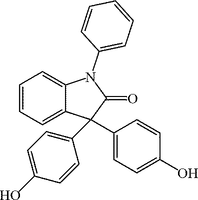 [SMILES: O=C1N(c2ccccc2)c2ccccc2C1(c1ccc(O)cc1)c1ccc(O)cc1]

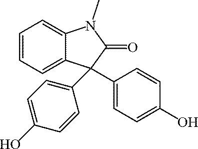 [SMILES: CN1C(=O)C(c2ccc(O)cc2)(c2ccc(O)cc2)c2ccccc21]

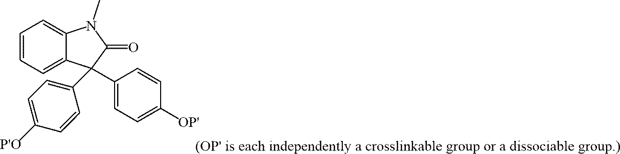

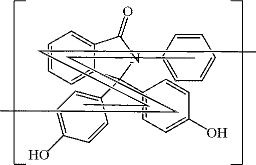

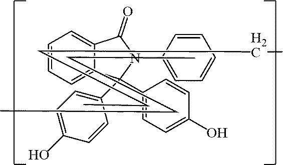

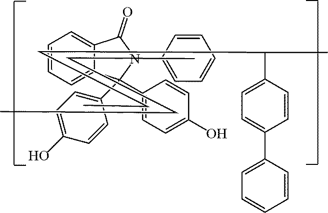

### Method for Producing Resin

In the resin of the present embodiment, as the method for polymerizing any one or more of the compound (4) to the compound (7) as monomers, a publicly known method can be used. For example, it can be obtained by electron oxidation polymerization in the presence of an oxidizing agent.

Also, in the resin (8) of the present embodiment, a publicly known method can also be used in the method for crosslinking using a compound with crosslinkability. For example, the resin can be obtained by using any one or more compounds among the compound (4) to the compound (7) as monomers and subjecting these monomers and a compound with crosslinking reactivity to a condensation reaction in the presence of an acid catalyst or in the presence of a base catalyst.

In the method for polymerizing any one or more of the compound (4) to the compound (7) as monomers, and in the condensation reaction using a compound with crosslinkability, a catalyst can also be used. The acid catalyst or base catalyst to be used here can be arbitrarily selected for use from publicly known catalysts. Examples of the acid catalyst include an organic acid and a solid acid. Specific examples thereof include an inorganic acid such as hydrochloric acid, sulfuric acid, phosphoric acid, hydrobromic acid, and hydrofluoric acid; an organic acid such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; a Lewis acid such as zinc chloride, aluminum chloride, iron chloride, and boron trifluoride; and a solid acid such as tungstosilicic acid, tungstophosphoric acid, silicomolybdic acid, and phosphomolybdic acid. Examples of the base catalyst include a metal alkoxide (for example, an alkali metal or alkaline earth metal alkoxide such as sodium methoxide, sodium ethoxide, potassium methoxide, and potassium ethoxide); a metal hydroxide (for example, an alkali metal or alkaline earth metal hydroxide such as sodium hydroxide and potassium hydroxide); an alkali metal or alkaline earth metal hydrogen carbonate such as sodium hydrogen carbonate and potassium hydrogen carbonate; an amine (for example, a tertiary amine (a trialkylamine such as triethylamine, an aromatic tertiary amine such as N,N-dimethylaniline, and a heterocyclic tertiary amine such as 1-methylimidazole) and the like; and an organic base of a metal carboxylate (for example, an alkali metal or alkaline earth metal acetate such as sodium acetate and calcium acetate). These catalysts are used alone as one kind or in combination of two or more kinds. As the catalyst, for example, an organic acid and a solid acid are preferable from the viewpoint of production, and hydrochloric acid or sulfuric acid is preferable from the viewpoint of production such as easy availability and handleability. The amount of the acid catalyst used can be arbitrarily set according to, for example, the kinds of the raw materials used and catalyst used, as well as the reaction conditions, and is preferably 0.01 to 100 parts by mass based on 100 parts by mass of the reaction raw materials, for example.

Note that, in the present embodiment, when the resin is produced through a copolymerization reaction with a compound having a nonconjugated double bond, such as indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, 5-vinylnorborn-2-ene, α-pinene, β-pinene, or limonene, and with a compound with crosslinking reactivity, it is not necessary to use the aldehyde or ketone.

In the method for polymerizing any one or more of the compound (4) to the compound (7) as monomers, and in the condensation reaction using a compound with crosslinkability, a reaction solvent can also be used. The reaction solvent in this polycondensation can be arbitrarily selected for use from publicly known solvents, and examples thereof include water, methanol, ethanol, propanol, butanol, 1-methoxy-2-propanol, tetrahydrofuran, dioxane, xylene such as ortho-xylene, and a mixed solvent thereof. These solvents are used alone as one kind, or in combination of two or more kinds.

The amount of the solvent used can be arbitrarily set according to, for example, the kinds of the raw materials used and catalyst used, as well as the reaction conditions, and is preferably in the range of 0 to 2000 parts by mass based on 100 parts by mass of the reaction raw materials, for example. Furthermore, the reaction temperature can be arbitrarily selected according to the reactivity of the reaction raw materials, and is usually in the range of 10 to 200° C. Note that examples of the reaction method include a method in which the compound (4) to the compound (7) and the oxidizing agent are fed in a batch, and a method in which these compounds and the oxidizing agent are fed successively. Examples thereof also include a method in which the compound (4) to the compound (7) and the catalyst are fed in a batch, and a method in which these compounds and the catalyst are fed successively. Examples thereof also include a method in which the compound (4) to the compound (7), the compound with crosslinkability such as the aldehyde and the ketone, and the catalyst are fed in a batch, and a method in which the compound (4) to the compound (7) and the compound with crosslinkability such as the aldehyde and/or the ketone are dripped successively in the presence of the catalyst.

After the polycondensation reaction terminates, isolation of the obtained resin can be carried out according to a conventional method. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the target compound (for example, the resin that has been made novolac) can be obtained.

### Solvent

The composition of the present embodiment may further contain a solvent. The solvent is not particularly limited as long as it is a solvent that can dissolve the compounds (1), (3) to (7) and the resin of the present embodiment, and various organic solvents can be suitably used.

Examples of the solvent include, but are not limited to, a ketone-based solvent such as acetone, methyl ethyl ketone, methyl isobutyl ketone, 2-heptanone, cyclopentanone (CPN), and cyclohexanone (CHN); a cellosolve-based solvent such as PGME (propylene glycol monomethyl ether) and PGMEA (propylene glycol monomethyl ether acetate); an ester-based solvent such as ethyl lactate, methyl acetate, ethyl acetate, butyl acetate, isoamyl acetate, ethyl lactate, methyl methoxypropionate, and methyl hydroxyisobutyrate; an alcohol-based solvent such as methanol, ethanol, isopropanol, and 1-ethoxy-2-propanol; and an aromatic hydrocarbon such as toluene, xylene, and anisole. These solvents are used alone as one kind or in combination of two or more kinds.

Among the above solvents, from the viewpoint of safety, it is preferable that the solvent be one or more selected from the group consisting of cyclohexanone, PGME, PGMEA, ethyl lactate, methyl hydroxyisobutyrate, and anisole when used in the first composition. When used in the second composition, the solvent is preferably a safe solvent, more preferably PGME, PGME, CHN, CPN, 2-heptanone, anisole, methyl hydroxyisobutyrate, butyl acetate, ethyl propionate, and ethyl lactate, and still more preferably PGMEA, PGME, and CHN. These solvents are used alone as one kind or in combination of two or more kinds. Note that the solvents described in a composition for resist film formation, which will be described later, can also be used as the solvent.

In the composition of the present embodiment, the amount of the solid components is preferably 1 to 80% by mass, more preferably 1 to 50% by mass, still more preferably 2 to 40% by mass, and even more preferably 2 to 10% by mass with 90 to 98% by mass of the solvent based on 100% by mass of the total mass of the solid components and the solvent but not particularly limited thereto.

In the composition of the present embodiment, the amount of the solvent is preferably 20 to 99% by mass, more preferably 50 to 99% by mass, still more preferably 60 to 98% by mass, and even more preferably 90 to 98% by mass based on 100% by mass of the total mass of the solid components and the solvent but not particularly limited thereto. In the present specification, “the solid components” refer to components except for the solvent.

The content of the solvent is not particularly limited, but from the viewpoint of solubility and film formation, it is preferably 100 to 10,000 parts by mass, more preferably 200 to 5,000 parts by mass, and still more preferably 200 to 1,000 parts by mass based on 100 parts by mass of the total mass of the compound (1) and the compound (3) in the first composition, or based on 100 parts by mass of the total mass of the compounds (4) to (7) and the resin in the second composition.

### Crosslinking Agent

The composition of the present embodiment may further contain a crosslinking agent from the viewpoint of, for example, suppressing intermixing. The crosslinking agent is not particularly limited, but those described in, for example, International Publication No. WO 2013/024778, International Publication No. WO 2013/024779, and International Publication No. WO 2018/016614 can be used.

Examples of the crosslinking agent include, but are not limited to, a phenol compound, an epoxy compound, a cyanate compound, an amino compound, a benzoxazine compound, an acrylate compound, a melamine compound, a guanamine compound, a glycoluril compound, a urea compound, an isocyanate compound, and an azide compound. These crosslinking agents are used alone as one kind or in combination of two or more kinds. In the first composition, among these, one or more selected from the group consisting of a benzoxazine compound, an epoxy compound, and a cyanate compound are preferable, and from the viewpoint of improvement in etching resistance, a benzoxazine compound is more preferable. In the second composition, among these, a melamine compound, a urea compound, a benzoxazine compound, an epoxy compound, and a cyanate compound are preferable, and from the point of having good reactivity, a melamine compound and a urea compound are more preferable. Examples of the melamine compound include a compound represented by formula (a) (NIKALAC MW-100LM (trade name), manufactured by Sanwa Chemical Co., Ltd.) and a compound represented by formula (b) (NIKALAC MX270 (trade name), manufactured by Sanwa Chemical Co., Ltd.).

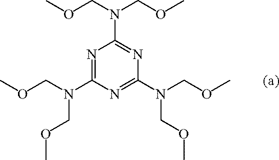

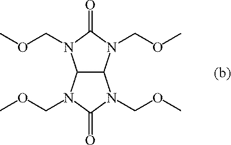

In the present embodiment, the content of the crosslinking agent is not particularly limited, but it is preferably 0.1 to 100 parts by mass, more preferably 5 to 50 parts by mass, and still more preferably 10 to 40 parts by mass based on 100 parts by mass of the total mass of the compound (1) and the compound (3) in the first composition, or based on 100 parts by mass of the total mass of the compounds (4) to (7) and the resin in the second composition. By setting the content of the crosslinking agent to the above range, occurrence of a mixing event with a resist film tends to be suppressed. Also, an antireflection effect is enhanced, and film formability after crosslinking tends to be enhanced.

### Crosslinking Promoting Agent

The composition of the present embodiment may further contain a crosslinking promoting agent for promoting the crosslinking reaction (curing reaction), if required. Examples of the crosslinking promoting agent include a radical polymerization initiator. Examples of the crosslinking promoting agent include the compounds disclosed in International Publication No. WO 2013/024778, International Publication No. WO 2013/024779, and International Publication No. WO 2017/033943.

The radical polymerization initiator may be a photopolymerization initiator that initiates radical polymerization by light, or may be a thermal polymerization initiator that initiates radical polymerization by heat. Examples of the radical polymerization initiator include, but are not limited to, a ketone-based photopolymerization initiator, an organic peroxide-based polymerization initiator, and an azo-based polymerization initiator.

Such a radical polymerization initiator is not particularly limited, but those described in, for example, International Publication No. WO 2018/016614 can be used.

These radical polymerization initiators are used alone as one kind or in combination of two or more kinds.

The content of the radical polymerization initiator in the present embodiment is not particularly limited, but it is preferably 0.05 to 25 parts by mass, and more preferably 0.1 to 10 parts by mass based on 100 parts by mass of the total mass of the compound (1) and the compound (3) in the first composition, or based on 100 parts by mass of the total mass of the compounds (4) to (7) and the resin in the second composition. When the content of the radical polymerization initiator is 0.05 parts by mass or more, there is a tendency to make it possible to prevent curing from being insufficient. On the other hand, when the content of the radical polymerization initiator is 25 parts by mass or less, there is a tendency to make it possible to prevent the long term storage stability at room temperature from being impaired.

### Acid Generating Agent

The composition of the present embodiment may further contain an acid generating agent from the viewpoint of, for example, further promoting the crosslinking reaction by heat. An acid generating agent that generates an acid by thermal decomposition, an acid generating agent that generates an acid by light irradiation, and the like are known, any of which can be used. The acid generating agent is not particularly limited, but those described in, for example, International Publication No. WO 2013/024778, International Publication No. WO 2013/024779, and International Publication No. WO 2017/033943 can be used.

The acid generating agent is preferably an acid generating agent having an aromatic ring, more preferably an acid generating agent having a sulfonic acid ion having an aryl group, and still more preferably di-tert-butyldiphenyliodonium nonafluoromethanesulfonate, diphenyltrimethylphenylsulfonium p-toluenesulfonate, triphenylsulfonium p-toluenesulfonate, triphenylsulfonium trifluoromethanesulfonate, and triphenylsulfonium nonafluoromethanesulfonate. By using the acid generating agent, line edge roughness can be reduced. These acid generating agents are used alone as one kind or in combination of two or more kinds.

The content of the acid generating agent in the composition of the present embodiment is not particularly limited, but it is preferably 0.1 to 50 parts by mass, and more preferably 0.5 to 40 parts by mass based on 100 parts by mass of the total mass of the compound (1) and the compound (3) in the first composition, or based on 100 parts by mass of the total mass of the compounds (4) to (7) and the resin in the second composition. By setting the content of the acid generating agent to the above range, the crosslinking reaction tends to be enhanced and occurrence of a mixing event with a resist film tends to be suppressed.

### Basic Compound

The composition of the present embodiment may further contain a basic compound from the viewpoint of, for example, improving storage stability.

The basic compound plays a role to suppress the crosslinking reaction from proceeding due to a trace amount of an acid generated from the acid generating agent, that is, a role as a quencher against the acid. Examples of such a basic compound include, but are not limited to, those described in, for example, International Publication No. WO 2013/024778, International Publication No. WO 2013/024779, and International Publication No. WO 2017/033943. These basic compounds are used alone as one kind or in combination of two or more kinds.

The content of the basic compound in the composition of the present embodiment is not particularly limited, but it is preferably 0.001 to 2 parts by mass, and more preferably 0.01 to 1 part by mass based on 100 parts by mass of the total mass of the compound (1) and the compound (3) in the first composition, or based on 100 parts by mass of the total mass of the compounds (4) to (7) and the resin in the second composition. By setting the content of the basic compound to the above range, storage stability tends to be enhanced without excessively deteriorating the crosslinking reaction.

### Acid Diffusion Controlling Agent

The film forming composition for lithography of the present embodiment may contain an acid diffusion controlling agent from the viewpoint of controlling diffusion of the acid generated from the acid generating agent by radiation irradiation in a resist film to inhibit any unpreferable chemical reaction in an unexposed region. By using the acid diffusion controlling agent, there is a tendency that the storage stability of the composition can be improved. Also, by using the acid diffusion controlling agent, there is a tendency that not only the resolution of a film formed by using the composition can be improved, but the line width change of a resist pattern due to variation in the post exposure delay time before radiation irradiation and the post exposure delay time after radiation irradiation can also be inhibited, making the composition excellent in process stability. Examples of the acid diffusion controlling agent include a radiation degradable basic compound such as a nitrogen atom-containing basic compound such as tributylamine and trioctylamine, a basic sulfonium compound, and a basic iodonium compound.

Examples of the acid diffusion controlling agent include the compounds disclosed in International Publication No. WO 2013/024778, International Publication No. WO 2013/024779, and International Publication No. WO 2017/033943. These acid diffusion controlling agents are used alone as one kind or in combination of two or more kinds.

The content of the acid diffusion controlling agent in the composition is preferably 0.001 to 49 parts by mass, more preferably 0.01 to 10 parts by mass, still more preferably 0.01 to 5 parts by mass, and even more preferably 0.01 to 3 parts by mass based on 100 parts by mass of the total mass of the compound (1) and the compound (3) in the first composition, or based on 100 parts by mass of the total of the compounds (4) to (7) and the resin in the second composition. When the amount of the acid diffusion controlling agent compounded is within the above range, there is a tendency that a decrease in resolution, and deterioration of the pattern shape and the dimension fidelity or the like can be prevented. Furthermore, even though the post exposure delay time from electron beam irradiation to heating after radiation irradiation becomes longer, the shape of the pattern upper layer portion can be suppressed from being deteriorated. Also, when the amount compounded is 10 parts by mass or less, there is a tendency that a decrease in sensitivity, and developability of the unexposed portion or the like can be prevented. By using such an acid diffusion controlling agent, there is a tendency that the storage stability of the composition is improved, the resolution is improved, and a good resist pattern is obtained.

### Additive

The composition of the present embodiment may further contain an additive other than the solvent, crosslinking agent, crosslinking promoting agent, acid generating agent, basic compound, and acid diffusion controlling agent described above, for the purpose of conferring thermosetting or light curing properties or controlling absorbance. Examples of such an additive include, but are not limited to, a naphthol resin, a xylene resin naphthol-modified resin, a phenol-modified resin of a naphthalene resin; a polyhydroxystyrene, a dicyclopentadiene resin, a resin containing (meth)acrylate, dimethacrylate, trimethacrylate, tetramethacrylate, a naphthalene ring such as vinylnaphthalene or polyacenaphthylene, a biphenyl ring such as phenanthrenequinone or fluorene, or a heterocyclic ring having a heteroatom such as thiophene or indene, and a resin containing no aromatic ring; and a resin or compound containing an alicyclic structure, such as a rosin-based resin, a cyclodextrin, an adamantine(poly)ol, a tricyclodecane(poly)ol, and a derivative thereof. The composition of the present embodiment may also contain a publicly known additive used for lithography film formation. Examples of the publicly known additive include, but are not limited to, a thermal and/or light curing catalyst, a polymerization inhibitor, a flame retardant, a filler, a coupling agent, a thermosetting resin, a light curable resin, a dye, a pigment, a thickener, a lubricant, an antifoaming agent, a leveling agent, an ultraviolet absorber, a surfactant, a colorant, and a nonionic surfactant.

### Composition for Resist Film Formation

It is preferable that the composition of the present embodiment be used for resist film formation. That is, a resist film of the present embodiment contains the composition of the present embodiment. A film formed by applying the composition of the present embodiment can also be used with a resist pattern formed, if required.

The composition of the present embodiment can be used as a film forming composition for lithography for chemical amplification type resist purposes (hereinafter, also referred to as “composition for resist film formation”). Hereinafter, components that can be contained in the composition for resist film formation will be described in particular.

In addition, it is preferable that the composition for resist film formation of the present embodiment contain a solvent. Examples of the solvent can include, but are not limited to, an ethylene glycol monoalkyl ether acetate such as ethylene glycol monomethyl ether acetate, ethylene glycol monoethyl ether acetate, ethylene glycol mono-n-propyl ether acetate, and ethylene glycol mono-n-butyl ether acetate; an ethylene glycol monoalkyl ether such as ethylene glycol monomethyl ether and ethylene glycol monoethyl ether; a propylene glycol monoalkyl ether acetate such as PGMEA, propylene glycol monoethyl ether acetate, propylene glycol mono-n-propyl ether acetate, and propylene glycol mono-n-butyl ether acetate; a propylene glycol monoalkyl ether such as PGME and propylene glycol monoethyl ether; a lactic acid ester such as methyl lactate, ethyl lactate, n-propyl lactate, n-butyl lactate, and n-amyl lactate; an aliphatic carboxylic acid ester such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutyl acetate, 3-methyl-3-methoxybutyl acetate, butyl 3-methoxy-3-methylpropionate, butyl 3-methoxy-3-methylbutyrate, methyl acetoacetate, methyl pyruvate, and ethyl pyruvate; an aromatic hydrocarbon such as toluene and xylene; a ketone such as 2-heptanone, 3-heptanone, 4-heptanone, cyclopentanone (hereinafter, also referred to as “CPN”), and cyclohexanone (hereinafter, also referred to as “CHN”); an amide such as N,N-dimethylformamide, N-methylacetamide, N,N-dimethylacetamide, and N-methylpyrrolidone; and a lactone such as γ-lactone. These solvents are used alone as one kind or in combination of two or more kinds.

The solvent used in the present embodiment is preferably a safe solvent, more preferably one or more selected from PGMEA, PGME, CHN, CPN, 2-heptanone, anisole, butyl acetate, ethyl propionate, and ethyl lactate, and still more preferably one or more selected from PGMEA, PGME, and CHN.

In the composition for resist film formation of the present embodiment, the amount of the solid components is preferably 1 to 80% by mass, more preferably 1 to 50% by mass, still more preferably 2 to 40% by mass, and even more preferably 2 to 10% by mass with 90 to 98% by mass of the solvent based on 100% by mass of the total mass of the solid components and the solvent but not particularly limited thereto.

In the composition for resist film formation of the present embodiment, the amount of the solvent is preferably 20 to 99% by mass, more preferably 50 to 99% by mass, still more preferably 60 to 98% by mass, and even more preferably 90 to 98% by mass based on 100% by mass of the total mass of the solid components and the solvent but not particularly limited thereto.

The composition for resist film formation of the present embodiment may further contain one or more selected from the group consisting of an acid generating agent, an acid crosslinking agent, an acid diffusion controlling agent, and an additional component, as solid components other than the compound (1), the compound (3), the compounds (4) to (7), and the resin of the present embodiment.

Here, as the acid generating agent, the acid crosslinking agent, the acid diffusion controlling agent, and the additional component, publicly known agents can be used, and they are not particularly limited, but those described in International Publication No. WO 2013/024778 are preferable, for example.

In the composition for resist film formation of the present embodiment, the total mass of the compound (1), the compound (3), the compounds (4) to (7), and the resin of the present embodiment used as a resist base material is preferably 1 to 100%, more preferably 50 to 99.4% by mass, still more preferably 55 to 90% by mass, even more preferably 60 to 80% by mass, and particularly preferably 60 to 70% by mass based on the entire mass of the solid components but not particularly limited thereto. When the total mass of the compound (1) and the compound (3) in the first composition or the total mass of the compounds (4) to (7) and the resin in the second composition is in the above range, there is a tendency that resolution is further improved and that line edge roughness (hereinafter, also referred to as “LER”) is further decreased.

The composition for resist film formation of the present embodiment, if required, may further contain various additives such as a dissolution promoting agent, a dissolution controlling agent, a sensitizing agent, a surfactant, an organic carboxylic acid or oxo acid of phosphor or a derivative thereof, a thermosetting catalyst, a light curing catalyst, a polymerization inhibitor, a flame retardant, a filler, a coupling agent, a thermosetting resin, a light curable resin, a dye, a pigment, a thickener, a lubricant, an antifoaming agent, a leveling agent, an ultraviolet absorber, a surfactant such as a nonionic surfactant, and a colorant within the range not inhibiting the objects of the present invention.

Note that these additives are used alone as one kind or in combination of two or more kinds.

In the composition for resist film formation of the present embodiment, the contents of: the compound (1), the compound (3); the compounds (4) to (7); the resin; the acid generating agent; the acid crosslinking agent; the acid diffusion controlling agent; and the additional component, of the present embodiment (compound (1) and compound (3), or compounds (4) to (7) and resin / acid generating agent / acid crosslinking agent / acid diffusion controlling agent / additional component) are, in terms of % by mass based on solid matter,
preferably 1 to 100 / 0 to 49 / 0 to 49 / 0 to 49 / 0 to 99,
more preferably 50 to 99.4 / 0.001 to 49 / 0.5 to 49 / 0.001 to 49 / 0 to 49,
still more preferably 55 to 90 / 1 to 40 / 0.5 to 40 / 0.01 to 10 / 0 to 5,
even more preferably 60 to 80 / 3 to 30 / 1 to 30 / 0.01 to 5 / 0 to 1, and
particularly preferably 60 to 70 / 10 to 25 / 2 to 20 / 0.01 to 3 / 0.

The content ratio of each component is selected from each range so that the summation thereof is 100% by mass. When the content ratio of each component falls within the above range, performance such as sensitivity, resolution, and developability tends to be excellent.

The composition for resist film formation of the present embodiment is generally prepared by dissolving each component in a solvent upon use into a homogeneous solution, and then if required, filtering through a filter or the like with a pore diameter of about 0.2 µm, for example.

The composition for resist film formation of the present embodiment can contain an additional resin other than the resin of the present embodiment, within the range not inhibiting the objects of the present embodiment. Examples of the additional resin include, but are not limited to, a novolac resin, a polyvinyl phenol, a polyacrylic acid, an epoxy resin, a polyvinyl alcohol, a styrene-maleic anhydride resin, and an addition polymerization resin.

The addition polymerization resin is not particularly limited, but examples thereof include a polymer containing an acrylic acid, a vinyl alcohol, a vinyl phenol, or a maleimide compound as a monomeric unit, and a derivative thereof.

The content of the additional resin is not particularly limited and is arbitrarily adjusted according the kinds of the compound (1), the compound (3), the compounds (4) to (7), and the resin of the present embodiment to be used, but it is preferably 30 parts by mass or less, more preferably 10 parts by mass or less, still more preferably 5 parts by mass or less, and even more preferably 0 parts by mass based on 100 parts by mass of the total mass of the compound (1) and the compound (3) in the first composition, or based on 100 parts by mass of the total mass of the compounds (4) to (7) and the resin in the second composition.

The composition for resist film formation of the present embodiment can be used to form an amorphous film by spin coating. Also, the composition for resist film formation of the present embodiment can be applied to a general semiconductor production process. Any of positive type and negative type resist patterns can be individually prepared depending on the kinds of the compound (1), the compound (3), the compounds (4) to (7), and the resin of the present embodiment and the kind of a developing solution to be used.

In the case of a positive type resist pattern, the dissolution rate of the amorphous film formed by spin coating with the composition for resist film formation of the present embodiment in a developing solution at 23° C. is preferably 5 angstrom/sec or less, more preferably 0.05 to 5 angstrom/sec, and further preferably 0.0005 to 5 angstrom/sec. When the dissolution rate is 5 angstrom/sec or less, there is a tendency of the amorphous film to be insoluble in a developing solution and therefore to easily form a resist. Also, when the dissolution rate is 0.0005 angstrom/sec or more, the resolution may be improved. It is presumed that this is because due to the change in the solubility before and after exposure of the compound (1), the compound (3), the compounds (4) to (7), and the resin of the present embodiment, contrast at the interface between the exposed portion being dissolved in a developing solution and the unexposed portion not being dissolved in a developing solution is increased. Also, effects of reducing LER and defects are confirmed.

In the case of a negative type resist pattern, the dissolution rate of the amorphous film formed by spin coating with the composition for resist film formation of the present embodiment in a developing solution at 23° C. is preferably 10 angstrom/sec or more. When the dissolution rate is 10 angstrom/sec or more, the amorphous film is more easily dissolved in a developing solution, and is suitable for a resist. Also, when the dissolution rate is 10 angstrom/sec or more, the resolution may be improved. It is presumed that this is because the micro surface portion of the compound (1), the compound (3), the compounds (4) to (7), and the resin of the present embodiment is dissolved, and LER is reduced. Also, effects of reducing defects are confirmed.

The above dissolution rate can be determined by immersing the amorphous film in a developing solution for a predetermined period of time at 23° C. and then measuring the film thickness before and after immersion by a publicly known method such as visual, ellipsometric, or QCM method.

In the case of a positive type resist pattern, the dissolution rate of the portion exposed by radiation such as KrF excimer laser, extreme ultraviolet, electron beam, or X-ray, of the amorphous film formed by spin coating with the composition for resist film formation of the present embodiment, in a developing solution at 23° C. is preferably 10 angstrom/sec or more. When the dissolution rate is 10 angstrom/sec or more, the above portion is more easily dissolved in a developing solution, and is suitable for a resist. Also, when the dissolution rate is 10 angstrom/sec or more, the resolution may be improved. It is presumed that this is because the micro surface portion of the compound (1), the compound (3), the compounds (4) to (7), and the resin of the present embodiment is dissolved and LER is reduced. Also, effects of reducing defects are confirmed.

In the case of a negative type resist pattern, the dissolution rate of the portion exposed by radiation such as KrF excimer laser, extreme ultraviolet, electron beam, or X-ray, of the amorphous film formed by spin coating with the composition for resist film formation of the present embodiment, in a developing solution at 23° C. is preferably 5 angstrom/sec or less, more preferably 0.05 to 5 angstrom/sec, and still more preferably 0.0005 to 5 angstrom/sec. When the dissolution rate is 5 angstrom/sec or less, there is a tendency of the above portion to be insoluble in a developing solution and therefore to easily form a resist. Also, when the dissolution rate is 0.0005 angstrom/sec or more, the resolution may be improved. It is presumed that this is because due to the change in the solubility before and after exposure of the compound (1), the compound (3), the compounds (4) to (7), and the resin of the present embodiment, contrast at the interface between the unexposed portion being dissolved in a developing solution and the exposed portion not being dissolved in a developing solution is increased. Also, effects of reducing LER and defects are confirmed.

The compound (1), the compound (3), the compounds (4) to (7), and the resin to be contained in the composition for resist film formation of the present embodiment are dissolved at preferably 1% by mass or more, more preferably 5% by mass or more, and still more preferably 10% by mass or more at 23° C. in a solvent that is selected from the group consisting of PGMEA, PGME, CHN, CPN, 2-heptanone, anisole, butyl acetate, ethyl propionate, and ethyl lactate and exhibits the highest ability to dissolve the compound (1), the compound (3), the compounds (4) to (7), and the resin.

The compound (1), the compound (3), the compounds (4) to (7), and the resin to be contained in the composition for resist film formation of the present embodiment are dissolved at preferably 20% by mass or more at 23° C. in a solvent that is selected from the group consisting of PGMEA, PGME, and CHN, and more preferably 20% by mass or more at 23° C. in PGMEA. When the above conditions are met, the composition is easily used in a semiconductor production process at a full production scale.

The composition for resist film formation of the present embodiment may contain an additional resin other than the present embodiment within the range not inhibiting the objects of the present embodiment. Examples of such an additional resin include a novolac resin, a polyvinyl phenol, a polyacrylic acid, a polyvinyl alcohol, a styrene-maleic anhydride resin, and a polymer containing an acrylic acid, a vinyl alcohol, or a vinylphenol as a monomeric unit, and a derivative thereof. The amount of these resins compounded is arbitrarily adjusted according the kinds of the compound (1), the compound (3), the compounds (4) to (7), and the resin of the present embodiment to be used, but it is preferably 30 parts by mass or less, more preferably 10 parts by mass or less, still more preferably 5 parts by mass or less, and particularly preferably 0 parts by mass based on 100 parts by mass of the total mass of the compound (1) and the compound (3) in the first composition, or based on 100 parts by mass of the total mass of the compounds (4) to (7) and the resin in the second composition.

### Resist Permanent Film

It is also preferable to use the composition of the present embodiment for forming a resist permanent film that remains in a final product, if required, with a resist pattern formed. That is, a resist permanent film of the present embodiment contains the composition of the present embodiment. A film formed by applying the composition of the present embodiment is suitable as a resist permanent film that remains also in a final product, if required, after formation of a resist pattern. Specific examples of the permanent film include, in relation to semiconductor devices, solder resists, package materials, underfill materials, package adhesive layers for circuit elements and the like, and adhesive layers between integrated circuit elements and circuit substrates, and in relation to thin displays, thin film transistor protecting films, liquid crystal color filter protecting films, black matrixes, and spacers. Particularly, the resist permanent film containing the composition of the present embodiment is excellent in heat resistance and humidity resistance, and furthermore, also has the excellent advantage that contamination by sublimable components is reduced. Particularly, for a display material, a material that achieves all of high sensitivity, high heat resistance, and hygroscopic reliability with reduced deterioration in image quality due to significant contamination can be obtained.

In the case of using the composition of the present embodiment for resist permanent film purposes, a curing agent, as well as, if required, various additives such as an additional resin, a surfactant, a dye, a filler, a crosslinking agent, and a dissolution promoting agent can be added and dissolved in an organic solvent to prepare a composition for resist permanent films.

### Composition for Resist Underlayer Film Formation

It is also preferable that the composition of the present embodiment be a composition used for resist underlayer film formation (hereinafter, also referred to as “composition for resist underlayer film formation”). That is, a resist underlayer film of the present embodiment contains the composition of the present embodiment.

For the composition for resist underlayer film formation of the present embodiment, all of the components described above as the components that can be contained by the composition for resist film formation can be applied in the same manner.

### Resist Pattern Formation Method

It is preferable that a resist pattern formation method of the present embodiment include: a resist underlayer film formation step of forming a resist underlayer film on a substrate using the composition for resist underlayer film formation of the present embodiment; a photoresist film formation step of forming at least one layer of photoresist film on the resist underlayer film; and a development step of irradiating a predetermined region of the photoresist film formed through the photoresist film formation step with radiation for development, thereby obtaining a resist pattern. Such a resist pattern formation method can be used for forming various patterns, and is preferably a method for forming an insulating film pattern.

In addition, it is also preferable that the resist pattern formation method of the present embodiment include: a photoresist layer formation step of forming a photoresist layer on the substrate using the composition for resist film formation of the present embodiment; and a development step of irradiating a predetermined region of the photoresist layer with radiation for development, thereby obtaining a resist pattern. Such a resist pattern formation method can also be used for forming various patterns, and is preferably a method for forming an insulating film pattern.

### Circuit Pattern Formation Method

A circuit pattern formation method of the present embodiment includes: a resist underlayer film formation step of forming a resist underlayer film on a substrate using the composition for resist underlayer film formation of the present embodiment; an intermediate layer film formation step of forming an intermediate layer film on the resist underlayer film; a photoresist film formation step of forming at least one layer of photoresist film on the intermediate layer film; a resist pattern formation step of irradiating a predetermined region of the photoresist film with radiation for development, thereby obtaining a resist pattern; an intermediate layer film pattern formation step of etching the intermediate layer film with the resist pattern as a mask, thereby obtaining an intermediate layer film pattern; a resist underlayer film pattern formation step of etching the resist underlayer film with the intermediate layer film pattern as a mask, thereby obtaining a resist underlayer film pattern; and a substrate pattern formation step of etching the substrate with the resist underlayer film pattern as a mask, thereby obtaining a substrate pattern.

A photoresist film and a resist underlayer film of the present embodiment are formed from the film forming composition for lithography of the present embodiment. The formation method therefor is not particularly limited and a publicly known method can be applied. The photoresist film and the resist underlayer film can be formed by, for example, applying the film forming composition for lithography of the present embodiment onto a substrate by a publicly known coating method or printing method such as spin coating or screen printing, and then removing an organic solvent by volatilization or the like.

It is preferable to perform baking in the formation of the resist underlayer film, for suppressing occurrence of a mixing event with a resist upper layer film while promoting the crosslinking reaction. In this case, the baking temperature is not particularly limited, but it is preferably in the range of 80 to 450° C., and more preferably 200 to 400° C. The baking time is not particularly limited as well, but it is preferably in the range of 10 to 300 seconds. Note that the thickness of the resist underlayer film can be arbitrarily selected according to required performance and is not particularly limited, but it is preferably 30 to 20,000 nm, and more preferably 50 to 15,000 nm.

After preparing the resist underlayer film, it is preferable to prepare a silicon-containing resist film or a single-layer resist made of hydrocarbon on the resist underlayer film in the case of a two-layer process, and to prepare a silicon-containing intermediate layer on the resist underlayer film and further prepare a silicon-free single-layer resist film on the silicon-containing intermediate layer in the case of a three-layer process. In this case, for a photoresist material for forming this resist film, a publicly known material can be used.

For the silicon-containing resist material for a two-layer process, a positive type photoresist material that is obtained by using, as a base polymer, a silicon atom-containing polymer such as a polysilsesquioxane derivative or a vinylsilane derivative and further includes an organic solvent, an acid generating agent, and if required, a basic compound and the like is preferably used from the viewpoint of etching resistance. Here, a publicly known polymer that is used in this kind of resist material can be used as the silicon atom-containing polymer.

A polysilsesquioxane-based intermediate layer is preferably used as the silicon-containing intermediate layer for a three-layer process. By imparting effects as an antireflection film to the intermediate layer, there is a tendency to make it possible to effectively suppress reflection. For example, use of a material containing a large amount of an aromatic group and having high substrate etching resistance as the resist underlayer film in a process for exposure at 193 nm tends to increase a k value and enhance substrate reflection. However, the intermediate layer suppresses the reflection so that the substrate reflection can be 0.5% or less. The intermediate layer having such an antireflection effect is not limited, but a polysilsesquioxane that crosslinks by an acid or heat in which a light absorbing group having a phenyl group or a silicon-silicon bond is introduced is preferably used for exposure at 193 nm.

Alternatively, an intermediate layer formed by chemical vapor deposition (CVD) may be used. The intermediate layer highly effective as an antireflection film prepared by CVD is not limited, and, for example, a SiON film is known. In general, the formation of an intermediate layer by a wet process such as spin coating or screen printing is more convenient and more advantageous in cost than CVD. Note that the upper layer resist for a three-layer process may be either positive type or negative type, and the same as a single-layer resist generally used can be used.

Furthermore, the resist underlayer film according to the present embodiment can also be used as an antireflection film for usual single-layer resists or an underlying material for suppression of pattern collapse. The resist underlayer film is excellent in etching resistance for an underlying process and can be expected to also function as a hard mask for an underlying process.

In the case of forming a photoresist film using the film forming composition for lithography of the present embodiment, a wet process such as spin coating or screen printing is preferably used, as in the case of forming the above resist underlayer film. Also, after coating with the resist material by spin coating or the like, prebaking is generally performed, and this prebaking is preferably performed at 80 to 180° C. in the range of 10 to 300 seconds. Then, exposure, post-exposure baking (PEB), and development can be performed according to a conventional method to obtain a resist pattern. The thickness of the resist film is not particularly limited, but in general, it is preferably 30 to 500 nm and more preferably 50 to 400 nm.

Exposure light can be arbitrarily selected for use according to the photoresist material to be used. General examples thereof can include a high energy ray having a wavelength of 300 nm or less, specifically, excimer laser of 248 nm, 193 nm, or 157 nm, soft X-ray of 3 to 20 nm, electron beam, and X-ray.

In a resist pattern formed by the method described above, pattern collapse is suppressed by the resist underlayer film. Therefore, use of the resist underlayer film of the present embodiment can produce a finer pattern and can reduce an exposure amount necessary for obtaining the resist pattern.

Next, etching is performed with the obtained resist pattern as a mask. Gas etching is preferably used as the etching of the resist underlayer film in a two-layer process. The gas etching is suitably etching using oxygen gas. In addition to oxygen gas, an inert gas such as He or Ar, or CO, CO₂, NH₃, SO₂, N₂, NO₂, or H₂ gas may be added. Alternatively, the gas etching may be performed with only CO, CO₂, NH₃, N₂, NO₂, or H₂ gas without the use of oxygen gas. Particularly, the latter gas is preferably used for side wall protection in order to prevent the undercut of pattern side walls.

On the other hand, gas etching is also preferably used as the etching of the intermediate layer in a three-layer process. The same gas etching as described in the above two-layer process is applicable. In particular, it is preferable to process the intermediate layer in a three-layer process by using chlorofluorocarbon-based gas and using the resist pattern as a mask. Then, as described above, for example, the resist underlayer film can be processed by oxygen gas etching with the intermediate layer pattern as a mask.

Here, in the case of forming an inorganic hard mask intermediate layer film as the intermediate layer, a silicon oxide film, a silicon nitride film, or a silicon oxynitride film (SiON film) is formed by CVD, ALD, or the like. A method for forming the nitride film is not particularly limited, and for example, a method described in Japanese Patent Laid-Open No. 2002-334869 (Patent Literature 9) or WO 2004/066377 (Patent Literature 10) can be used. Although a photoresist film can be formed directly on such an intermediate layer film, an organic antireflection film (BARC) may be formed on the intermediate layer film by spin coating and a photoresist film may be formed thereon.

A polysilsesquioxane-based intermediate layer is also suitably used as the intermediate layer. By imparting effects as an antireflection film to the resist intermediate layer film, there is a tendency to make it possible to effectively suppress reflection. A specific material for the polysilsesquioxane-based intermediate layer is not limited, and, for example, a material described in Japanese Patent Laid-Open No. 2007-226170 (Patent Literature 11) or Japanese Patent Laid-Open No. 2007-226204 (Patent Literature 12) can be used.

The subsequent etching of the substrate can also be performed by a conventional method. For example, the substrate made of SiO₂ or SiN can be etched mainly using chlorofluorocarbon-based gas, and the substrate made of p-Si, Al, or W can be etched mainly using chlorine- or bromine-based gas. In the case of etching the substrate with chlorofluorocarbon-based gas, the silicon-containing resist of the two-layer resist process or the silicon-containing intermediate layer of the three-layer process is stripped at the same time with substrate processing. On the other hand, in the case of etching the substrate with chlorine- or bromine-based gas, the silicon-containing resist film or the silicon-containing intermediate layer is separately stripped and in general, stripped by dry etching using chlorofluorocarbon-based gas after substrate processing.

A feature of the resist underlayer film of the present embodiment is that it is excellent in etching resistance of the substrate. Note that the substrate can be arbitrarily selected for use from publicly known ones and is not particularly limited. Examples thereof include Si, α-Si, p-Si, SiO₂, SiN, SiON, W, TiN, and Al. The substrate may be a laminate having a film to be processed (substrate to be processed) on a base material (support). Examples of such a film to be processed include, but are not limited to, various low-k films such as Si, SiO₂, SiON, SiN, p-Si, α-Si, W, W-Si, Al, Cu, and Al-Si, and stopper films thereof. A material different from that for the base material (support) is generally used. The thickness of the substrate to be processed or the film to be processed is not particularly limited, but it is generally preferably about 50 to 1,000,000 nm, and more preferably 75 to 50,000 nm.

The composition of the present embodiment can be prepared by adding each of the components to be contained and mixing them using a stirrer or the like. When the composition of the present embodiment contains a filler or a pigment, it can be prepared by dispersion or mixing using a dispersion apparatus such as a dissolver, a homogenizer, and a three-roll mill.

### EXAMPLES

The present embodiment will be described in more detail with reference to examples below. However, the present embodiment is not limited to these examples by any means.

### Measurement of Mn, Mw, and Mw/Mn

The number average molecular weight (Mn), mass average molecular weight (Mw), and dispersity (Mw/Mn) were determined in terms of polystyrene by gel permeation chromatography (GPC) analysis under the following measurement conditions.
Apparatus: “Shodex GPC-101 model” (product name, manufactured by Showa Denko K.K.)
Column: “KF-80M” × 3 (product name, manufactured by Showa Denko K.K.)
Eluent: tetrahydrofuran (hereinafter, also referred to as “THF”)
Flow rate: 1 mL/min
Temperature: 40° C.

### (Synthesis Working Example 1) Synthesis of R-BINL-2EO

A four necked flask (internal capacity: 1 L) equipped with a Dimroth condenser tube, a thermometer, and a stirring blade and having a detachable bottom was prepared. To this four necked flask, 26.3 g (50 mmol) of 2,2′-bis(2-hydroxyethoxy)-6,6′-diphenyl1,1′-binaphthalene (hereinafter, abbreviated as “BINL-2EO”), 21.0 g (280 mmol as formaldehyde) of a 40 mass% aqueous formaldehyde solution (manufactured by Mitsubishi Gas Chemical Company, Inc.), and 0.97 mL of a 98 mass% sulfuric acid (manufactured by Kanto Chemical Co., Inc.) were added in a nitrogen stream, and the mixture was allowed to react for 7 hours while being refluxed at 100° C. at normal pressure. Subsequently, 180.0 g of ortho-xylene (special grade reagent manufactured by Wako Pure Chemical Industries, Ltd.) was added as a diluting solvent to the reaction liquid, and the mixture was left to stand still, followed by removal of an aqueous phase as a lower phase. Neutralization and washing with water were further performed, and ortho-xylene was distilled off under reduced pressure to obtain 18.0 g of a brown solid resin (R-BINL-2EO).

Here, BINL-2EO was synthesized by the same method as the synthesis method described in paragraph 0062 of International Publication No. WO 2019/044875.

As a result of measuring the Mw and Mw/Mn of the obtained resin (R-BINL-2EO) by the above method, Mw = 1300 and Mw/Mn = 1.30.

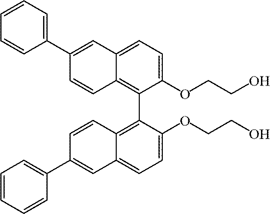 [SMILES: OCCOc1ccc2cc(-c3ccccc3)ccc2c1-c1c(OCCO)ccc2cc(-c3ccccc3)ccc12]

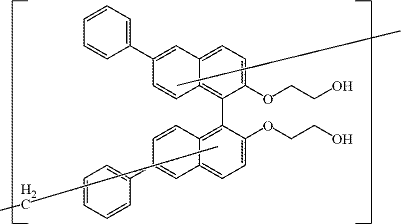

### (Synthesis Working Example 2) Synthesis of R2-BINL-2EO

Synthesis was performed in the same manner as in Synthesis Working Example 1 using 9.2 g (50 mmol) of 4-biphenylaldehyde (manufactured by Mitsubishi Gas Chemical Company, Inc.) instead of using 21.0 g (280 mmol as formaldehyde) of a 40 mass% aqueous formaldehyde solution (manufactured by Mitsubishi Gas Chemical Company, Inc.), thereby obtaining the objective compound (R2-BINL-2EO) represented by formula (R2-BINL-2EO) below.

As a result of measuring the Mw and Mw/Mn of the obtained resin (R2-BINL-2EO) by the above method, Mw = 1410 and Mw/Mn = 1.40.

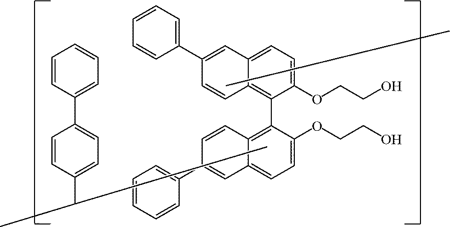

### (Synthesis Working Example 3) Synthesis of R3-BINL-2EO

To a container (internal capacity: 500 mL) equipped with a stirrer, a condenser tube, and a burette, 26.3 g (50 mmol) of BINL-2EO and 5 g (10 mmol) of monobutyl phthalate copper were added, and 100 mL of 1-butanol was added as a solvent. The reaction liquid was allowed to react while stirring at 100° C. for 6 hours. After cooling, the precipitates were filtered and the resulting crude was dissolved in 100 mL of ethyl acetate. Next, 5 mL of hydrochloric acid was added, and the mixture was stirred at room temperature, and neutralized with sodium hydrogen carbonate. The ethyl acetate solution was concentrated and 200 mL of methanol was added to precipitate the reaction product. After cooling to room temperature, the precipitates were separated by filtration. The obtained solid matter was dried to obtain 19 g of the objective resin (R3-BINL-2EO) having a structure represented by the formula below.

The polystyrene equivalent molecular weight of the obtained resin was measured by the method described above, and as a result, Mn = 920 and Mw/Mn = 1.25.

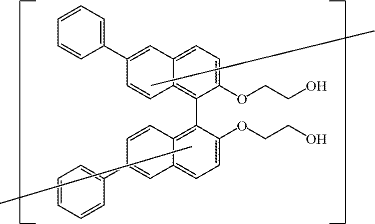

### (Synthesis Comparative Example 1) Synthesis of C-1

A four necked flask (internal capacity: 10 L) equipped with a Dimroth condenser tube, a thermometer, and a stirring blade and having a detachable bottom was prepared. To this four necked flask, 1.09 kg (7 mol) of 1,5-dimethylnaphthalene (manufactured by Mitsubishi Gas Chemical Company, Inc.), 2.1 kg (28 mol as formaldehyde) of a 40 mass% aqueous formalin solution (manufactured by Mitsubishi Gas Chemical Company, Inc.), and 0.97 ml of a 98 mass% sulfuric acid (manufactured by Kanto Chemical Co., Inc.) were added in a nitrogen stream, and the mixture was allowed to react for 7 hours while being refluxed at 100° C. at normal pressure. Subsequently, 1.8 kg of ethylbenzene (manufactured by Wako Pure Chemical Industries, Ltd., special grade reagent) was added as a diluting solvent to the reaction liquid, and the mixture was left to stand still, followed by removal of an aqueous phase as a lower phase. Neutralization and washing with water were further performed, and ethylbenzene and unreacted 1,5-dimethylnaphthalene were distilled off under reduced pressure to obtain 1.25 kg of a dimethylnaphthalene formaldehyde resin as a light brown solid. The molecular weight of the obtained dimethylnaphthalene formaldehyde resin was as follows: number average molecular weight (Mn): 562, weight average molecular weight (Mw): 1168, and dispersity (Mw/Mn): 2.08.

Subsequently, a four necked flask (internal capacity: 0.5 L) equipped with a Dimroth condenser tube, a thermometer, and a stirring blade was prepared. To this four necked flask, 100 g (0.51 mol) of the dimethylnaphthalene formaldehyde resin obtained as described above and 0.05 g of p-toluenesulfonic acid were added in a nitrogen stream, and the temperature was raised to 190° C. at which the mixture was then heated for 2 hours, followed by stirring. Subsequently, 52.0 g (0.36 mol) of 1-naphthol was further added thereto, and the temperature was further raised to 220° C. at which the mixture was allowed to react for 2 hours. After dilution with a solvent, neutralization and washing with water were performed, and the solvent was distilled off under reduced pressure to obtain 126.1 g of resin (C-1) as a black-brown solid.

The obtained resin (C-1) had Mn: 885, Mw: 2220, and Mw/Mn: 2.51.

### (Synthesis Example 1) Synthesis of AC-1

4.15 g of 2-methyl-2-methacryloyloxyadamantane, 3.00 g of methacryloyloxy-γ-butyrolactone, 2.08 g of 3-hydroxy-1-adamantyl methacrylate, and 0.38 g of azobisisobutyronitrile were dissolved in 80 mL of tetrahydrofuran to prepare a reaction solution. This reaction solution was polymerized for 22 hours with the reaction temperature kept at 63° C. in a nitrogen atmosphere. Then, the reaction solution was added dropwise into 400 mL of n-hexane. The obtained product resin was solidified and purified, and the resulting white powder was filtered and then dried overnight at 40° C. under reduced pressure to obtain AC-1 represented by the formula below.

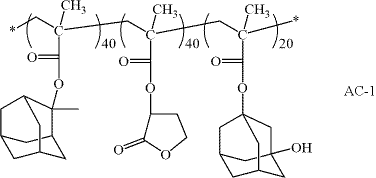

In formula AC-1, “40”, “40”, and “20” represent the ratio of each constituent unit and do not indicate that the resin is a block copolymer.

### Performance Evaluation of Composition for Resist Film Formation: Examples 1-1 to 5-1 and Comparative Example 1

Each of the compositions for resist film formation with compositional features shown in Table 1 below was prepared using 6,6′-diphenyl-1,1′-bis-2,2′naphthol (hereinafter, abbreviated as “BINL”), BINL-2EO, which is the same as used in Synthesis Working Example 1, and R-BINL-2EO obtained in Synthesis Working Example 1, and R2-BINL-2EO and R3-BINL-2EO obtained in Synthesis Working Examples 2 and 3, respectively, as the compound. The following acid generating agent, acid diffusion controlling agent, and organic solvent were used. Acid generating agent: triphenylsulfonium nonafluoromethanesulfonate manufactured by Midori Kagaku Co., Ltd. (denoted in the table as “TPS-109”.); acid diffusion controlling agent: tri-n-octylamine manufactured by Kanto Chemical Co., Inc. (denoted in the table as “TOA”.); crosslinking agent: NIKALAC MW-100LM manufactured by Sanwa Chemical Co., Ltd. (denoted in the table as “MW-100LM”.); and organic solvent: propylene glycol monomethyl ether manufactured by Kanto Chemical Co., Inc. (denoted in the table as “PGME”.)

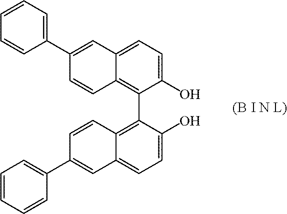 [SMILES: Oc1ccc2cc(-c3ccccc3)ccc2c1-c1c(O)ccc2cc(-c3ccccc3)ccc12]

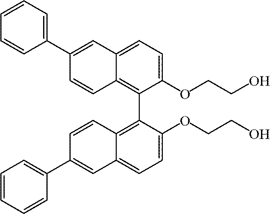 [SMILES: OCCOc1ccc2cc(-c3ccccc3)ccc2c1-c1c(OCCO)ccc2cc(-c3ccccc3)ccc12]

TABLE 1

| | | | | | |
|---|---|---|---|---|---|
| | Compound or resin (parts by mass) | Acid generating agent (parts by mass) | Acid diffusion controlling agent (parts by mass) | Crosslinking agent (parts by mass) | Organic solvent (parts by mass) |
| Example 1-1 | BINL (5) | TPS-109 (1) | TOA (0.1) | MW-100LM (1) | PGME (100) |
| Example 2-1 | BINL-2EO (5) | TPS-109 (1) | TOA (0.1) | MW-100LM (1) | PGME (100) |
| Example 3-1 | R-BINL-2EO (5) | TPS-109 (1) | TOA (0.1) | MW-100LM (1) | PGME (100) |
| Example 4-1 | R2-BINL-2EO (5) | TPS-109 (1) | TOA (0.1) | MW-100LM (1) | PGME (100) |
| Example 5-1 | R3-BINL-2EO (5) | TPS-109 (1) | TOA (0.1) | MW-100LM (1) | PGME (100) |
| Comparative Example 1 | C-1 (5) | TPS-109 (1) | TOA (0.1) | MW-100LM (1) | PGME (100) |

### Evaluation Methods

### Safe Solvent Solubility Test of Compound or Resin

The solubility of the compounds or resins in PGME, PGMEA, and CHN was evaluated from the amount of dissolution in each solvent using the following criteria. Note that the amount of dissolution was measured at 23° C. by precisely weighing the compound or resin alone into a test tube, adding the target solvent so as to attain a predetermined concentration, applying ultrasonic waves for 30 minutes in an ultrasonic cleaner, and then visually observing the subsequent state of the fluid. A: 5.0% by mass ≤ amount of dissolution; B: 2.0% by mass ≤ amount of dissolution < 5.0% by mass; and C: amount of dissolution < 2.0% by mass

### Storage Stability of Composition for Resist Film Formation

The storage stability of the compositions for resist film formation containing the compounds or resins was evaluated by leaving the compositions for resist film formation after preparation to stand still at 23° C. for 3 days, and visually observing the presence or absence of precipitates. Also, a clean silicon wafer was spin coated with the compositions for resist film formation, and then pre-exposure baked (PB) on a hot plate at 110° C. to form a resist film with a thickness of 50 nm. The compositions for resist film formation were evaluated as C if there were precipitates, evaluated as B if the solution was homogeneous but there were defects in the thin film, and evaluated as A if the solution was homogeneous, there were no defects in the thin film, and the thin film formation was good.

### Resist Pattern and Sensitivity

The resist films obtained in the above (2) were irradiated with electron beams of 1:1 line and space setting with a 50 nm interval using an electron beam lithography system (ELS-7500 manufactured by ELIONIX INC.). After such irradiation, the resist films were each heated at 110° C. for 90 seconds, and immersed in a 2.38 mass% TMAH alkaline developing solution for 60 seconds for development. Subsequently, the resist films were washed with ultrapure water for 30 seconds, and dried to form resist patterns. The shape of the obtained resist patterns of 50 nm L/S (1:1) was observed using an electron microscope manufactured by Hitachi Ltd. (S-4800). The shape of the resist patterns after development was evaluated as A when having better rectangularity compared to Comparative Example 1 without pattern collapse, and evaluated as C when it is equivalent to or inferior to Comparative Example 1. Furthermore, the smallest electron beam energy quantity capable of lithographing good pattern shapes was evaluated as the sensitivity. That is, one that is superior to Comparative Example 1 by 10% or more was evaluated as A, one that is superior but by less than 10% was evaluated as B, and one that is equivalent to or inferior to Comparative Example 1 was evaluated as C.

### Etching Resistance

Etching apparatus: RIE-10NR manufactured by Samco International, Inc.; Output: 50 W; Pressure: 20 Pa; Time: 2 min; and etching gas: Ar gas flow rate:CF₄ gas flow rate:O₂ gas flow rate = 50:5:5 (sccm). For the resist films obtained in the above (2), the etching test was carried out using the etching apparatus and under the conditions described above, and the etching rate upon that time was measured. Then, the etching resistance was evaluated according to the following evaluation criteria on the basis of the etching rate of a resist film prepared by using a novolac (“PSM4357” manufactured by Gunei Chemical Industry Co., Ltd.). A: The etching rate was less than -15% as compared with the resist film of novolac; B: The etching rate was -15% to +5% as compared with the resist film of novolac; and C: The etching rate was more than +5% as compared with the resist film of novolac.

For each of the compounds or resins used in Examples 1-1 to 5-1 and Comparative Example 1, the evaluation results of solubility in safe solvents by the method described above are shown in Table 2.

Furthermore, the evaluation results by the respective methods described above for the compositions for resist film formation of Examples 1-1 to 5-1 and Comparative Example 1 are shown in Table 2.

TABLE 2

| | Solubility of compound or resin | | | Evaluation results | | | |
|---|---|---|---|---|---|---|---|
| | PGME | PGMEA | CHN | Storage stability and thin film formation | Resist pattern | Sensitivity | Etching resistance |
| Example 1-1 | A | A | A | ○ | A | A | A |
| Example 2-1 | A | A | A | ○ | A | A | A |
| Example 3-1 | A | A | A | ○ | A | A | A |
| Example 4-1 | A | A | A | ○ | A | A | A |
| Example 5-1 | A | A | A | ○ | A | A | A |
| Comparative Example 1 | A | A | A | ○ | C | C | C |

### Performance Evaluation of Composition for Resist Underlayer Film formation Examples 1-2 to 5-2 and Comparative Example 2

Each of the compositions for resist underlayer film formation with compositional features shown in Table 3 was prepared. Next, a silicon substrate was spin coated with these compositions for resist underlayer film formation, and then baked at 240° C. for 60 seconds and further at 400° C. for 120 seconds to prepare each resist underlayer film with a film thickness of 200 nm. The following acid generating agent, crosslinking agent, and organic solvent were used.
Acid generating agent: di-tert-butyl diphenyliodonium nonafluoromethanesulfonate (denoted in the table as “DTDPI”.) (manufactured by Midori Kagaku Co., Ltd.)
Crosslinking agent: “NIKALAC MX270” (denoted in the table as “MX270”.) (product name, manufactured by Sanwa Chemical Co., Ltd.)
Organic solvent: propylene glycol monomethyl ether acetate (also referred to as “PGMEA” in the table)

Then, by the same method as described in the above (4) Etching resistance, the etching resistance of the compositions for resist underlayer film formation of Examples 1-2 to 5-2 and Comparative Example 2 was evaluated. The results are also shown in Table 3.

TABLE 3

| | | | | | |
|---|---|---|---|---|---|
| | Compound or resin (parts by mass) | Acid generating agent (parts by mass) | Crosslinking agent (parts by mass) | Organic solvent (parts by mass) | Etching resistance |
| Example 1-2 | BINL-2EO (10) | DTDPI (0.5) | MX270 (0.5) | PGME (90) | A |
| Example 2-2 | BINL-2EO (10) | DTDPI (0.5) | MX270 (0.5) | PGME (90) | A |
| Example 3-2 | R-BINL-2EO (10) | DTDPI (0.5) | MX270 (0.5) | PGME (90) | A |
| Example 4-2 | R2-BINL-2EO (10) | DTDPI (0.5) | MX270 (0.5) | PGME (90) | A |
| Example 5-2 | R3-BINL-2EO (10) | DTDPI (0.5) | MX270 (0.5) | PGME (90) | A |
| Comparative Example 2 | C-2 (10) | DTDPI (0.5) | MX270 (0.5) | PGME (100) | C |

### Examples 1-3 to 5-3

A SiO₂ substrate with a film thickness of 300 nm was coated with each of the compositions for resist underlayer film formation prepared in the above Examples 1-3 to 5-3, and baked at 240° C. for 60 seconds and further at 400° C. for 120 seconds to form resist underlayer films with a film thickness of 70 nm. These resist underlayer films were coated with a resist solution for ArF and baked at 130° C. for 60 seconds to form photoresist films with a film thickness of 140 nm. Note that the ArF resist solution used was prepared by compounding 5 parts by mass of the resin (AC-1) of Synthesis Example, 1 part by mass of triphenylsulfonium nonafluoromethanesulfonate, 2 parts by mass of tributylamine, and 92 parts by mass of PGMEA.

Subsequently, the photoresist films were exposed using an electron beam lithography system “ELS-7500” (product name, manufactured by ELIONIX INC., 50 keV), baked (PEB) at 115° C. for 90 seconds, and developed for 60 seconds in a 2.38 mass% tetramethylammonium hydroxide (TMAH) aqueous solution to obtain positive type resist patterns.

Defects of the obtained resist patterns of 55 nm L/S (1:1) and 80 nm L/S (1:1) were observed, and the respective results are shown in Table 4. In the table, “Good” shown as a result of “Resist pattern after development” indicates that no pattern collapse was observed in the formed resist pattern, and “Poor” indicates that pattern collapse was observed in the formed resist pattern. Also, the smallest line width having good rectangularity without pattern collapse as a result of the above observation was used as an index for “Resolution” evaluation. Furthermore, the smallest electron beam energy quantity capable of lithographing good pattern shapes was used as an index for “Sensitivity” evaluation. The results are shown in Table 4.

### Comparative Example 3

The same operations as in Example 1-3 were carried out except that no underlayer film was formed so that a photoresist film was formed directly on a SiO₂ substrate to obtain a positive type resist pattern. The results are shown in Table 4.

TABLE 4

| | Composition for resist underlayer film formation | Resolution (nmL/S) | Sensitivity (µC/cm²) | Resist pattern after development |
|---|---|---|---|---|
| Example 1-3 | Example 1-2 | 55 | 10 | Good |
| Example 2-3 | Example 2-2 | 55 | 10 | Good |
| Example 3-3 | Example 3-2 | 55 | 10 | Good |
| Example 4-3 | Example 3-2 | 55 | 10 | Good |
| Example 5-3 | Example 3-2 | 55 | 10 | Good |
| Comparative Example 3 | None | 80 | 26 | Poor |

### Examples 1-4 to 5-4

A SiO₂ substrate with a film thickness of 300 nm was coated with each of the compositions for resist underlayer film formation of Examples 1-4 to 5-4, and baked at 240° C. for 60 seconds and further at 400° C. for 120 seconds to form resist underlayer films with a film thickness of 80 nm. These resist underlayer films were coated with a silicon-containing intermediate layer material and baked at 200° C. for 60 seconds to form intermediate layer films with a film thickness of 35 nm. These intermediate layer films were further coated with the above resist solution for ArF and baked at 130° C. for 60 seconds to form photoresist films with a film thickness of 150 nm. Note that the silicon-containing intermediate layer material used was the silicon atom-containing polymer described in <Synthesis Example 1> of Japanese Patent Laid-Open No. 2007-226170. Subsequently, the photoresist films were mask exposed using an electron beam lithography system (manufactured by ELIONIX INC.; ELS-7500, 50 keV), baked (PEB) at 115° C. for 90 seconds, and developed for 60 seconds in a 2.38 mass% tetramethylammonium hydroxide (hereinafter, also referred to as “TMAH”) aqueous solution to obtain 55 nm L/S (1:1) positive type resist patterns. Thereafter, the silicon-containing intermediate layer films were dry etched with the obtained resist patterns as a mask using a parallel plate RIE system “RIE-10NR” (trade name, manufactured by Samco International, Inc.). Subsequently, dry etching of the resist underlayer films with the obtained silicon-containing intermediate layer film patterns as a mask and dry etching of the SiO₂ film with the obtained resist underlayer film patterns as a mask were performed in order.

The respective etching conditions are as shown below.
Conditions for etching of resist intermediate layer film with resist pattern
Output: 50 W
Pressure: 20 Pa
Time: 1 min
Etching gas
Ar gas flow rate:CF₄ gas flow rate:O₂ gas flow rate = 50:8:2 (sccm) Conditions for etching of resist underlayer film with resist intermediate film pattern
Output: 50 W
Pressure: 20 Pa
Time: 2 min
Etching gas
Ar gas flow rate:CF₄ gas flow rate:O₂ gas flow rate = 50:5:5 (sccm)
Conditions for etching of SiO₂ film with resist underlayer film pattern Output: 50 W
Pressure: 20 Pa
Time: 2 min
Etching gas
Ar gas flow rate:C₅F₁₂ gas flow rate:C₂F₆ gas flow rate:O₂ gas flow rate
= 50:4:3:1 (sccm)

The pattern cross section (that is, the shape of the SiO₂ film after etching) obtained as described above was observed by using an electron microscope “S-4800” (product name, manufactured by Hitachi, Ltd.) to evaluate resist pattern formability. The observation results are shown in Table 5. In the table, “Good” shown as “Resist pattern formability” indicates that no major defects were found in the formed pattern cross section, and “Poor” indicates that major defects were found in the formed pattern cross section.

TABLE 5

| | Composition for resist underlayer film formation | Thickness of SiO₂ film (nm) | Resist pattern formability |
|---|---|---|---|
| Example 1-4 | Example 1-2 | 10 | Good |
| Example 2-4 | Example 2-2 | 10 | Good |
| Example 3-4 | Example 3-2 | 10 | Good |
| Example 4-4 | Example 3-2 | 10 | Good |
| Example 5-4 | Example 3-2 | 10 | Good |

As is clear from the above, the compositions of the present embodiment simultaneously satisfy solubility in organic solvents, etching resistance, and resist pattern formability at high levels for lithography film formation, and are useful for lithography film formation.

### (Synthesis Working Example A1) Synthesis of R-PPPBP

A four necked flask (internal capacity: 1 L) equipped with a Dimroth condenser tube, a thermometer, and a stirring blade and having a detachable bottom was prepared. To this four necked flask, 19.7 g (50 mmol) of compound (PPPBP) obtained by the method described in Synthesis Example 1 of International Publication No. WO 2011/090022, 21.0 g (280 mmol as formaldehyde) of a 40 mass% aqueous formaldehyde solution (manufactured by Mitsubishi Gas Chemical Company, Inc.), and 0.97 mL of a 98 mass% sulfuric acid (manufactured by Kanto Chemical Co., Inc.) were added in a nitrogen stream, and the mixture was allowed to react for 7 hours while being refluxed at 100° C. at normal pressure. Subsequently, 180.0 g of ortho-xylene (special grade reagent manufactured by Wako Pure Chemical Industries, Ltd.) was added as a diluting solvent to the reaction liquid, and the mixture was left to stand still, followed by removal of an aqueous phase as a lower phase. Neutralization and washing with water were further performed, and ortho-xylene was distilled off under reduced pressure to obtain 12.8 g of a brown solid resin (R-PPPBP).

The obtained resin (R-PPPBP) had Mw: 1570 and Mw/Mn: 1.35.

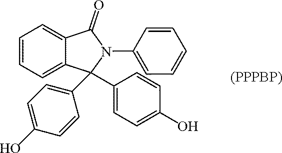 [SMILES: O=C1c2ccccc2C(c2ccc(O)cc2)(c2ccc(O)cc2)N1c1ccccc1]

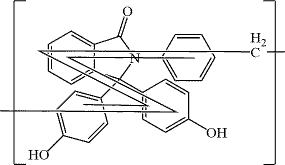

### (Synthesis Working Example A2) Synthesis of R2-PPPBP

Synthesis was performed in the same manner as in Synthesis Working Example 1 using 9.2 g (50 mmol) of 4-biphenylaldehyde (manufactured by Mitsubishi Gas Chemical Company, Inc.) instead of using 21.0 g (280 mmol as formaldehyde) of a 40 mass% aqueous formaldehyde solution (manufactured by Mitsubishi Gas Chemical Company, Inc.), thereby obtaining the objective compound (R2-PPPBP) represented by formula (R2-PPPBP) below.

As a result of measuring the Mw and Mw/Mn of the obtained resin (R2-PPPBP) by the above method, Mw = 1600 and Mw/Mn = 1.45.

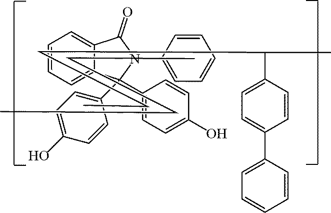

### (Synthesis Working Example A3) Synthesis of R3-PPPBP

To a container (internal capacity: 500 mL) equipped with a stirrer, a condenser tube, and a burette, 19.7 g (50 mmol) of PPPBP and 5 g (10 mmol) of monobutyl phthalate copper were added, and 100 mL of 1-butanol was added as a solvent. The reaction liquid was allowed to react while stirring at 100° C. for 6 hours. After cooling, the precipitates were filtered and the resulting crude was dissolved in 100 mL of ethyl acetate. Next, 5 mL of hydrochloric acid was added, and the mixture was stirred at room temperature, and neutralized with sodium hydrogen carbonate. The ethyl acetate solution was concentrated and 200 mL of methanol was added to precipitate the reaction product. After cooling to room temperature, the precipitates were separated by filtration. The obtained solid matter was dried to obtain the objective resin (R3-PPPBP) having a structure represented by the formula below.

The polystyrene equivalent molecular weight of the obtained resin was measured by the method described above, and as a result, Mn = 1100 and Mw/Mn = 1.30.

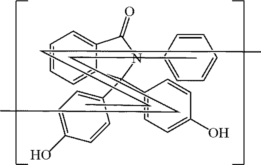

### (Synthesis Comparative Example A1) Synthesis of C-1

A four necked flask (internal capacity: 10 L) equipped with a Dimroth condenser tube, a thermometer, and a stirring blade and having a detachable bottom was prepared. To this four necked flask, 1.09 kg (7 mol) of 1,5-dimethylnaphthalene (manufactured by Mitsubishi Gas Chemical Company, Inc.), 2.1 kg (28 mol as formaldehyde) of a 40 mass% aqueous formalin solution (manufactured by Mitsubishi Gas Chemical Company, Inc.), and 0.97 ml of a 98 mass% sulfuric acid (manufactured by Kanto Chemical Co., Inc.) were added in a nitrogen stream, and the mixture was allowed to react for 7 hours while being refluxed at 100° C. at normal pressure.

Subsequently, 1.8 kg of ethylbenzene (manufactured by Wako Pure Chemical Industries, Ltd., special grade reagent) was added as a diluting solvent to the reaction liquid, and the mixture was left to stand still, followed by removal of an aqueous phase as a lower phase. Neutralization and washing with water were further performed, and ethylbenzene and unreacted 1,5-dimethylnaphthalene were distilled off under reduced pressure to obtain 1.25 kg of a dimethylnaphthalene formaldehyde resin as a light brown solid.

The obtained dimethylnaphthalene formaldehyde resin had Mn: 562, Mw: 1168, and Mw/Mn: 2.08.

Subsequently, a four necked flask (internal capacity: 0.5 L) equipped with a Dimroth condenser tube, a thermometer, and a stirring blade was prepared. To this four necked flask, 100 g (0.51 mol) of the dimethylnaphthalene formaldehyde resin obtained as described above and 0.05 g of p-toluenesulfonic acid were added in a nitrogen stream, and the temperature was raised to 190° C. at which the mixture was then heated for 2 hours, followed by stirring. Subsequently, 52.0 g (0.36 mol) of 1-naphthol was further added thereto, and the temperature was further raised to 220° C. at which the mixture was allowed to react for 2 hours. After dilution with a solvent, neutralization and washing with water were performed, and the solvent was distilled off under reduced pressure to obtain 126.1 g of resin (C-1) as a black-brown solid. Note that, for the resin (C-1), representative substructures are shown below. These substructures are bonded via methylene groups, but some are also bonded via ether bonds and the like.

The obtained resin (C-1) had Mn: 885, Mw: 2220, and Mw/Mn: 2.51.

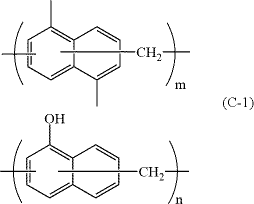

### (Synthesis Example A1) Synthesis of AC-1

4.15 g of 2-methyl-2-methacryloyloxyadamantane, 3.00 g of methacryloyloxy-y-butyrolactone, 2.08 g of 3-hydroxy-1-adamantyl methacrylate, and 0.38 g of azobisisobutyronitrile were dissolved in 80 mL of tetrahydrofuran to prepare a reaction solution. This reaction solution was polymerized for 22 hours with the reaction temperature kept at 63° C. in a nitrogen atmosphere. Then, the reaction solution was added dropwise into 400 mL of n-hexane. The obtained resin was solidified and purified, and the resulting white powder was filtered and then dried overnight at 40° C. under reduced pressure to obtain AC-1 represented by the formula below.

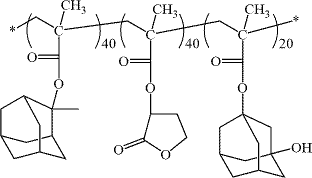

In formula AC-1, “40”, “40”, and “20” represent the ratio of each constituent unit and do not indicate that the resin is a block copolymer.

### Performance Evaluation of Composition for Resist Film Formation

### Examples A1-1 to A5-1 and Comparative Example A1

Each of the compositions for lithography film formation (compositions for resist film formation) with compositional features shown in Table 6 was prepared using the compound (PPPBP, the structural formula is as described above) obtained by the method described in Synthesis Example 1 of International Publication No. WO 2011/090022, the compound (BisP-IST-NMe) obtained by the method described in Synthesis Example 5 of International Publication No. WO 2011/090022, the resins (R-PPPBP, R2-PPPBP, and R3-PPPBP) obtained by Synthesis Working Examples A1 to A4, and the resin (C-1) obtained by Synthesis Comparative Example A1. Note that the following acid generating agent, acid diffusion controlling agent, crosslinking agent, and organic solvent were used. Also, in Table 6, the numerical values in parentheses indicate the amount compounded (parts by mass).
Acid generating agent: triphenylsulfonium trifluoromethanesulfonate (TPS-109 (trade name), manufactured by Midori Kagaku Co., Ltd.)
Acid diffusion controlling agent: tri-n-octylamine (TOA, manufactured by Kanto Chemical Co., Inc.)
Crosslinking agent: NIKALAC MW-100LM (MW-100LM, trade name, manufactured by Sanwa Chemical Co., Ltd.)
Organic solvent: propylene glycol monomethyl ether (PGME, manufactured by Kanto Chemical Co., Inc.)

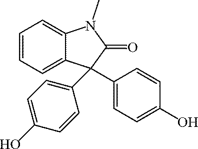 [SMILES: CN1C(=O)C(c2ccc(O)cc2)(c2ccc(O)cc2)c2ccccc21]

TABLE 6

| | | | | | |
|---|---|---|---|---|---|
| | Compound or resin (parts by mass) | Acid generating agent (parts by mass) | Acid diffusion controlling agent (parts by mass) | Crosslinking agent (parts by mass) | Organic solvent (parts by mass) |
| Example A1-1 | PPPBP (5) | TPS-109 (1) | TOA (0.1) | MW-100LM (1) | PGME (100) |
| Example A2-1 | BisP-IST-NMe (5) | TPS-109 (1) | TOA (0.1) | MW-100LM (1) | PGME (100) |
| Example A3-1 | R-PPPBP (5) | TPS-109 (1) | TOA (0.1) | MW-100LM (1) | PGME (100) |
| Example A4-1 | R2-PPPBP (5) | TPS-109 (1) | TOA (0.1) | MW-100LM (1) | PGME (100) |
| Example A5-1 | R3-PPPBP (5) | TPS-109 (1) | TOA (0.1) | MW-100LM (1) | PGME (100) |
| Comparative Example A1 | C-1 (5) | TPS-109 (1) | TOA (0.1) | MW-100LM (1) | PGME (100) |

### Evaluation Methods

### Solubility in Safe Solvents

The solubility of each of the compound (PPPBP), the compound (BisP-IST-NMe), the resins (R-PPPBP, R2-PPPBP, R3-PPPBP, and R4-PPPBP), and the resin (C-1) in PGME (manufactured by Kanto Chemical Co., Inc.), PGMEA (manufactured by Kanto Chemical Co., Inc.), and CHN (manufactured by Kanto Chemical Co., Inc.) was evaluated. Specifically, the solubility was evaluated from the amount of dissolution in each solvent using the following criteria. Note that the amount of dissolution was measured at 23° C. by precisely weighing the compound or resin alone into a test tube, adding the target solvent so as to attain a predetermined concentration, applying ultrasonic waves for 30 minutes in an ultrasonic cleaner, and then visually observing the subsequent state of the fluid.
A: 5.0% by mass ≤ amount of dissolution
B: 2.0% by mass ≤ amount of dissolution < 5.0% by mass
C: amount of dissolution < 2.0% by mass

### Storage Stability and Thin Film Formation of Composition for Resist Film Formation

The storage stability of the compositions for resist film formation was evaluated by, after preparation according to the compositional features described in Table 6, leaving the respective compositions for resist film formation to stand still at 23° C. for 3 days, and visually observing the presence or absence of precipitates. The compositions for resist film formation after being left to stand still for 3 days was evaluated as ◯ if the solution was homogeneous and there were no precipitates, and evaluated as × if there were precipitates. Also, a clean silicon wafer was spin coated with the homogeneous compositions for resist film formation, and then pre-exposure baked (PB) in an oven at 110° C. to form a resist film with a thickness of 40 nm. The prepared resist film was evaluated as ◯ if the thin film formability was good, and evaluated as × if the formed film had defects.

### Resist Pattern

The respective resist films obtained in the above evaluation method (2) were irradiated with electron beams of 1:1 line and space setting with a 50 nm interval using an electron beam lithography system (ELS-7500, manufactured by ELIONIX INC., 50 keV).

After the irradiation, the resist films were each heated at 110° C. for 90 seconds, and immersed in a 2.38 mass% tetramethylammonium hydroxide (TMAH) alkaline developing solution for 60 seconds for development. Subsequently, the respective resist films were washed with ultrapure water for 30 seconds, and dried to form resist patterns.

The shape of the obtained resist patterns of L/S (1:1) with a 50 nm interval was observed using an electron microscope manufactured by Hitachi Ltd. (S-4800, trade name). The shape of the resist patterns after development was evaluated as A when having better rectangularity compared to Comparative Example A1 without pattern collapse, and evaluated as C when it is equivalent to or inferior to Comparative Example A1. Note that, in Comparative Example A1, pattern collapse was observed in the resist pattern shape after development, and the rectangularity was poor.

Furthermore, the sensitivity was evaluated using the smallest electron beam energy quantity capable of lithographing good pattern shapes in a stepwise manner. That is, one whose smallest electron beam energy quantity is superior to that of Comparative Example A1 by 10% or more was evaluated as S, one whose smallest electron beam energy quantity is superior to that of Comparative Example A1 but by less than 10% was evaluated as A, and one that is equivalent to or inferior to Comparative Example A1 was evaluated as C.

### Etching Resistance

For each of the resist films obtained in the evaluation method (2) described above, the etching test was carried out under the following conditions, and the etching rate upon that time was measured. In addition, a resist film was prepared by the same method as described in the evaluation method (2) described above, using a composition obtained by using a novolac resin (PSM4357 (model number) manufactured by Gunei Chemical Industry Co., Ltd.) instead of PPPBP of Example A1-1 in the compositional features shown in Table 6, and the etching test was carried out on this resist film as well under the following conditions. The etching resistance of each resist film was evaluated according to the following evaluation criteria on the basis of the etching rate of the resist film obtained by using the novolac resin.

### (Etching Conditions)

Etching apparatus: RIE-10NR (trade name) manufactured by Samco International, Inc.
Output: 50 W
Pressure: 20 Pa
Time: 2 min
Etching gas: Ar gas flow rate:CF₄ gas flow rate:O₂ gas flow rate = 50:5:5 (sccm)

### (Evaluation Criteria)

A: The etching rate was less than -15% as compared with the resist film of the novolac resin.
B: The etching rate was -15% to +5% as compared with the resist film of the novolac resin.
C: The etching rate was more than +5% as compared with the resist film of the novolac resin.

Their evaluation results are shown in Table 7. Note that, in Table 7, for the solubility of each of the compound (PPPBP), the compound (BisP-IST-NMe), the resin (R-PPPBP), and the resin (C-1) in each solvent, their results are shown in Example A1-1, Example A2-1, Example A3-1, and Comparative Example A1, respectively.

TABLE 7

| | Solubility of compound or resin | | | Evaluation results | | | |
|---|---|---|---|---|---|---|---|
| | PGM E | PGME A | CHN | Storage stability and thin film formatio n | Resist pattern | Sensit ivity | Etching resistance |
| Example A1-1 | A | A | A | ○ | A | S | A |
| Example A2-1 | A | A | A | ○ | A | S | A |
| Example A3-1 | A | A | A | ○ | A | S | A |
| Example A4-1 | A | A | A | ○ | A | S | A |
| Example A5-1 | A | A | A | ◯ | A | S | A |
| Comparative Example A1 | A | A | A | ○ | C | C | C |

### Performance Evaluation of Composition for Resist Underlayer Film Formation

### Examples A1-2 to A5-2 and Comparative Example 2

Using the compound (PPPBP), the compound (BisP-IST-NMe), the resins (R-PPPBP, R2-PPPBP, and R3-PPPBP), and the resin (C-1), the respective compositions for lithography film formation (compositions for resist underlayer film formation) with compositional features shown in Table 8 were prepared. Note that the following acid generating agent, crosslinking agent, and organic solvent were used. Also, in Table 8, the numerical values in parentheses indicate the amount compounded (parts by mass).
Acid generating agent: di-tert-butyl diphenyliodonium nonafluoromethanesulfonate (DTDPI, manufactured by Midori Kagaku Co., Ltd.)
Crosslinking agent: NIKALAC MX270 (MX270, trade name, manufactured by Sanwa Chemical Co., Ltd.)
Organic solvent: propylene glycol monomethyl ether acetate (PGMEA, manufactured by Kanto Chemical Co., Inc.)

Subsequently, a silicon substrate was spin coated with each of these compositions for resist underlayer film formation or a novolac resin (PSM4357 (trade name) manufactured by Gunei Chemical Industry Co., Ltd.), and then heated at 240° C. for 60 seconds and further baked at 400° C. for 120 seconds to prepare each resist underlayer film with a film thickness of 200 nm.

For each of the obtained resist underlayer films, the etching test was carried out under the etching conditions described in the evaluation method (4) described above. In addition, a composition obtained by using the novolac resin instead of PPPBP of Example A1-2 in the compositional features shown in Table 8 was prepared. On the basis of the etching rate of a resist underlayer film obtained by using this composition, the etching resistance of each resist film was evaluated according to the following evaluation criteria.

### (Evaluation Criteria)

A: The etching rate was less than -15% as compared with the resist underlayer film of the novolac resin.
B: The etching rate was -15% to +5% as compared with the resist underlayer film of the novolac resin.
C: The etching rate was more than +5% as compared with the resist underlayer film of the novolac resin.

Their evaluation results are shown in Table 8.

TABLE 8

| | | | | | |
|---|---|---|---|---|---|
| | Compound or resin (parts by mass) | Acid generating agent (parts by mass) | Crosslinking agent (parts by mass) | Organic solvent (parts by mass) | Etching resistance |
| Example A1-2 | PPPBP (10) | DTDPI (0.5) | MX270 (0.5) | PGME (90) | A |
| Example A2-2 | BisP-IST-NMe (10) | DTDPI (0.5) | MX270 (0.5) | PGME (90) | A |
| Example A3-2 | R-PPPBP (10) | DTDPI (0.5) | MX270 (0.5) | PGME (90) | A |
| Example A4-2 | R2-PPPBP (10) | DTDPI (0.5) | MX270 (0.5) | PGME (90) | A |
| Example A5-2 | R3-PPPBP (10) | DTDPI (0.5) | MX270 (0.5) | PGME (90) | A |
| Comparative Example A2 | C-2 (10) | DTDPI (0.5) | MX270 (0.5) | PGME (100) | C |

### Examples A1-3 to A5-3

A SiO₂ substrate with a film thickness of 300 nm was coated with each of the compositions for resist underlayer film formation prepared in Examples A1-2 to A5-2, and heated at 240° C. for 60 seconds and further baked at 400° C. for 120 seconds to form resist underlayer films with a film thickness of 70 nm. These resist underlayer films were coated with resist solution A for ArF excimer laser and baked at 130° C. for 60 seconds to form photoresist films with a film thickness of 140 nm. Note that the resist solution A for ArF excimer laser used was prepared by compounding 5 parts by mass of the resin (AC-1) obtained in Synthesis Example A1, 1 part by mass of triphenylsulfonium trifluoromethanesulfonate (TPS-109 (trade name), manufactured by Midori Kagaku Co., Ltd.), 2 parts by mass of tributylamine (manufactured by Kanto Chemical Co., Inc.), and 92 parts by mass of PGMEA (manufactured by Kanto Chemical Co., Inc.).

Next, using an electron beam lithography system (ELS-7500, manufactured by ELIONIX INC., 50 keV), each of the obtained photoresist films formed on the resist underlayer films was irradiated with electron beams of 1:1 line and space setting with 55 nm and 80 nm intervals and exposed. Thereafter, the photoresist films were baked (PEB) at 115° C. for 90 seconds, and immersed in a 2.38 mass% tetramethylammonium hydroxide alkaline developing solution for 60 seconds for development, thereby obtaining positive type resist patterns (1).

Using the obtained resist patterns of L/S (1:1) with a 55 nm interval and resist patterns of L/S (1:1) with an 80 nm interval, their respective defects were observed using an electron microscope (S-4800 (trade name), manufactured by Hitachi, Ltd.). The results are shown in Table 9. Note that, in Table 9, “Good” indicates that no pattern collapse was observed in the formed resist pattern, and “Poor” indicates that pattern collapse was observed in the formed resist pattern.

Furthermore, the smallest electron beam energy quantity capable of lithographing good pattern shapes was measured and evaluated as the sensitivity.

### Comparative Example A3

Positive type resist patterns were obtained in the same manner as in Examples A1-3 to A5-3 except that no resist underlayer film was formed and a photoresist film was directly formed on a SiO₂ substrate with a film thickness of 300 nm, using the resist solution A for ArF excimer laser. Thereafter, in the same manner as in Examples A1-3 to A5-3, using the obtained resist pattern of L/S (1:1) with a 55 nm interval and resist pattern of L/S (1:1) with an 80 nm interval, their respective defects were observed using an electron microscope (S-4800 (trade name), manufactured by Hitachi, Ltd.).

Furthermore, the smallest electron beam energy quantity capable of lithographing good pattern shapes was measured, and evaluated as the sensitivity.

The results are shown in Table 9.

TABLE 9

| | Composition for resist underlayer film formation | Resolution (nmL/S) | Sensitivity (µC/cm²) | Resist pattern after development |
|---|---|---|---|---|
| Example A1-3 | Example A1-2 | 55 | 10 | Good |
| Example A2-3 | Example A2-2 | 55 | 10 | Good |
| Example A3-3 | Example A3-2 | 55 | 10 | Good |
| Example A4-3 | Example A4-2 | 55 | 10 | Good |
| Example A5-3 | Example A5-2 | 55 | 10 | Good |
| Comparative Example A3 | None | 80 | 26 | Poor |

### Examples A1-4 to A5-4

A SiO₂ substrate with a film thickness of 300 nm was coated with each of the compositions for resist underlayer film formation prepared in Examples A1-2 to A5-2, and heated at 240° C. for 60 seconds and further baked at 400° C. for 120 seconds to form resist underlayer films with a film thickness of 80 nm. These resist underlayer films were coated with a silicon-containing intermediate layer material and baked at 200° C. for 60 seconds to form silicon-containing intermediate layer films with a film thickness of 35 nm. Furthermore, these silicon-containing intermediate layer films were coated with the resist solution A for ArF excimer laser described above and baked at 130° C. for 60 seconds to form photoresist films with a film thickness of 150 nm. Note that the silicon-containing intermediate layer material used was the silicon atom-containing polymer described in <Synthesis Example 1> of Japanese Patent Laid-Open No. 2007-226170.

Next, using an electron beam lithography system (ELS-7500, manufactured by ELIONIX INC., 50 keV), each of the obtained photoresist films formed on the silicon-containing intermediate layer films was irradiated with electron beams of 1:1 line and space setting with a 55 nm interval and exposed to the shape to be used as a mask during dry etching. Thereafter, the photoresist films were baked (PEB) at 115° C. for 90 seconds, and immersed in a 2.38 mass% tetramethylammonium hydroxide alkaline developing solution for 60 seconds for development, thereby obtaining positive type resist patterns of L/S (1:1) with a 55 nm interval.

Thereafter, the silicon-containing intermediate layer films were dry etched with the obtained resist patterns as a mask using an etching apparatus (parallel plate RIE system, RIE-10NR (trade name), manufactured by Samco International, Inc.) under the following conditions. Subsequently, dry etching of the resist underlayer films with the obtained silicon-containing intermediate layer film patterns as a mask, and then, dry etching of the SiO₂ film with the obtained resist underlayer film patterns as a mask were performed.

The respective etching conditions are as shown below.
Conditions for etching of silicon-containing intermediate layer film Output: 50 W
   Pressure: 20 Pa
   Time: 1 min
   Etching gas: Ar gas flow rate:CF₄ gas flow rate:O₂ gas flow rate = 50:8:2 (sccm)
Conditions for etching of resist underlayer film
   Output: 50 W
   Pressure: 20 Pa
   Time: 2 min
   Etching gas: Ar gas flow rate:CF₄ gas flow rate:O₂ gas flow rate = 50:5:5 (sccm)
Conditions for etching of SiO₂ film
   Output: 50 W
   Pressure: 20 Pa
   Time: 2 min
   Etching gas: Ar gas flow rate:C₅F₁₂ gas flow rate:C₂F₆ gas flow rate:O₂ gas flow rate = 50:4:3:1 (sccm)

The pattern cross section (that is, the shape of the SiO₂ substrate after etching) obtained as described above was observed by using an electron microscope (S-4800 (trade name), manufactured by Hitachi, Ltd.) to evaluate resist pattern formability. The observation results are shown in Table 10. In the table, “Good” indicates that no major defects were found in the formed pattern cross section, and “Poor” indicates that major defects were found in the formed pattern cross section.

TABLE 10

| | Composition for resist underlayer film formation | Thickness of SiO2 film (nm) | Resist pattern formability |
|---|---|---|---|
| Example A1-4 | Example A1-2 | 10 | Good |
| Example A2-4 | Example A2-2 | 10 | Good |
| Example A3-4 | Example A3-2 | 10 | Good |
| Example A4-4 | Example A4-2 | 10 | Good |
| Example A5-4 | Example A5-2 | 10 | Good |

As indicated by Tables 6 to 10, according to the present embodiment, it is possible to provide a composition that is useful as a film forming material for lithography, having high solubility in organic solvents, excellent storage stability and thin film formability, high etching resistance, high sensitivity, and excellent resist pattern formability, and satisfying a good balance of these physical properties at high levels.

The present application is based on Japanese Patent Application No. 2020-118023 filed in the Japan Patent Office on Jul. 8, 2020 and Japanese Patent Application No. 2020-135055 filed in the Japan Patent Office on Aug. 7, 2020, the contents of which are incorporated herein by reference.

### INDUSTRIAL APPLICABILITY

The first composition has high heat resistance, has high solvent solubility, and is applicable to a wet process. Also, according to the second composition, it is possible to provide a composition that is useful as a film forming material for lithography, having high solubility in organic solvents, excellent storage stability and thin film formability, high etching resistance, high sensitivity, and excellent resist pattern formability, and satisfying a good balance of these physical properties at high levels. In addition, the composition of the present invention has excellent heat resistance and high solubility in solvents, and is thus suitable for a wet process. Therefore, a film forming material for lithography using the composition of the present invention, and a film for lithography thereof can be utilized widely and effectively in various applications that require such performances. Accordingly, the present invention can be utilized widely and effectively in, for example, electrical insulating materials, resins for resists, encapsulation resins for semiconductors, adhesives for printed circuit boards, electrical laminates mounted in electric equipment, electronic equipment, industrial equipment, and the like, matrix resins of prepregs mounted in electric equipment, electronic equipment, industrial equipment, and the like, buildup laminate materials, resins for fiber-reinforced plastics, resins for encapsulation of liquid crystal display panels, coating materials, various coating agents, adhesives, coating agents for semiconductors, resins for resists for semiconductors, resins for resist underlayer film formation, and the like. In particular, the present invention can be utilized particularly effectively in the field of films for lithography.

1. A film forming composition for lithography comprising at least one selected from: a compound represented by general formula (1); a compound represented by general formula (3); a compound represented by formula (4); a compound represented by formula (5); and a resin obtained using the compound represented by formula (4) or the compound represented by formula (5) as a monomer, 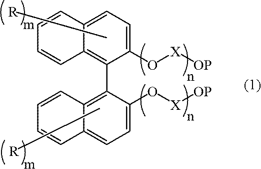
wherein
R each independently represents an aromatic group having 6 to 36 carbon atoms and optionally having a substituent or a heteroatom;
X each independently represents an alkanediyl group having 2 to 4 carbon atoms or alkanediylcarbonyl group having 1 to 4 carbon atoms, each of which optionally has a substituent;
P each independently represents an alkyl group having 1 to 30 carbon atoms, aryl group having 6 to 30 carbon atoms, alkenyl group having 2 to 20 carbon atoms, or alkynyl group having 2 to 20 carbon atoms, each of which optionally has a substituent, or a hydrogen atom, a crosslinkable group, or a dissociable group; and
m each independently represents an integer of 1 to 6 and n each independently represents an integer of 0 to 4, 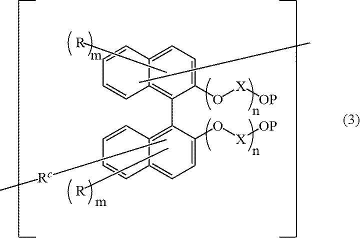
wherein
R each independently represents an aromatic group having 6 to 36 carbon atoms and optionally having a substituent or a heteroatom;
R^{c} each independently represents a single bond, a linear or branched alkylene group having 1 to 20 carbon atoms and optionally having a substituent, or an arylene group having 1 to 20 carbon atoms and optionally having a substituent;
X each independently represents an alkanediyl group having 2 to 4 carbon atoms or alkanediylcarbonyl group having 1 to 4 carbon atoms, each of which optionally has a substituent;
P each independently represents an alkyl group having 1 to 30 carbon atoms, aryl group having 6 to 30 carbon atoms, alkenyl group having 2 to 20 carbon atoms, or alkynyl group having 2 to 20 carbon atoms, each of which optionally has a substituent, or a hydrogen atom, a crosslinkable group, or a dissociable group; and
m each independently represents an integer of 1 to 6 and n each independently represents an integer of 0 to 4, 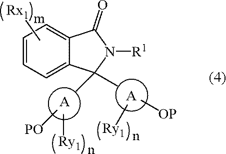
wherein
A each independently represents an aromatic group having 6 to 10 carbon atoms;
P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group;
R¹ represents an aromatic substituent having 6 to 10 carbon atoms or an alkyl group having 1 to 20 carbon atoms;
Rx₁ each independently represents a hydrogen atom, an alkyl group having 1 to 20 carbon atoms, an alkoxy group having 1 to 20 carbon atoms, or a halogen;
Ry₁ each independently represents a hydrogen atom, an alkyl group having 1 to 20 carbon atoms, an alkoxy group having 1 to 20 carbon atoms, or a halogen; and
m represents an integer of 0 to 4 and n represents an integer of 0 to 4, and 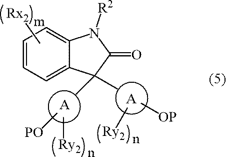
wherein
A each independently represents an aromatic group having 6 to 10 carbon atoms;
P each independently represents a hydrogen atom, an alkyl group having 1 to 30 carbon atoms, an aryl group having 6 to 30 carbon atoms, an alkenyl group having 2 to 20 carbon atoms, an alkynyl group having 2 to 20 carbon atoms, a crosslinkable group, or a dissociable group;
R² represents an aromatic substituent having 6 to 10 carbon atoms or an alkyl group having 1 to 20 carbon atoms;
Rx₂ each independently represents a hydrogen atom, an alkyl group having 1 to 20 carbon atoms, an alkoxy group having 1 to 20 carbon atoms, or a halogen;
Ry₂ each independently represents a hydrogen atom, an alkyl group having 1 to 20 carbon atoms, an alkoxy group having 1 to 20 carbon atoms, or a halogen; and
m represents an integer of 0 to 4 and n represents an integer of 0 to 4.

2. The film forming composition for lithography according to claim 1, wherein the compound represented by general formula (1) is a compound represented by general formula (2), 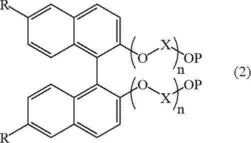
wherein R, X, P, and n are the same as defined in formula (1).

3. The film forming composition for lithography according to claim 1, comprising the compound represented by general formula (3).

4. The film forming composition for lithography according to claim 1, comprising at least one selected from: the compound represented by formula (4); the compound represented by formula (5); and the resin obtained using the compound represented by formula (4) or the compound represented by formula (5) as a monomer.

5. The film forming composition for lithography according to claim 4, wherein
the compound represented by formula (4) is a compound represented by formula (6); and
the compound represented by formula (5) is a compound represented by formula (7), 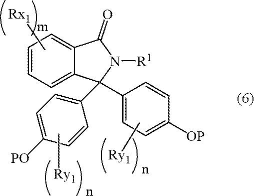
wherein
P, R¹, Rx₁ Ry₁, m, and n are the same as defined in formula (4), and 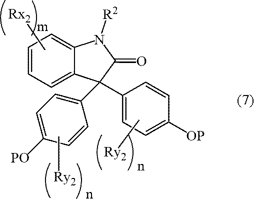
wherein
P, R², Rx₂ Ry₂, m, and n are the same as defined in formula (5).

6. The film forming composition for lithography according to claim 4, wherein the resin is a resin represented by formula (8), 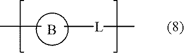
wherein
B is a constituent unit derived from the compound represented by formula (4) and/or a constituent unit derived from the compound represented by formula (5);
L is a single bond, a linear or branched alkylene group having 1 to 20 carbon atoms and optionally having a substituent, or an arylene group having 1 to 20 carbon atoms and optionally having a substituent; and
when there are a plurality of B and/or L, they are independent of each other.

7. The film forming composition for lithography according to claim 1, further comprising a solvent.

8. The film forming composition for lithography according to claim 1, further comprising an acid generating agent.

9. The film forming composition for lithography according to claim 1, further comprising a crosslinking agent.

10. A resist pattern formation method comprising:
a photoresist layer formation step of forming a photoresist layer on a substrate using the film forming composition for lithography according to claim 1; and
a development step of irradiating a predetermined region of the photoresist layer with radiation for development, thereby obtaining a resist pattern.

11. The resist pattern formation method according to claim 10, wherein the resist pattern is an insulating film pattern.

12. A resist pattern formation method, comprising:
a resist underlayer film formation step of forming a resist underlayer film on a substrate using the film forming composition for lithography according to claim 1;
a photoresist layer formation step of forming at least one layer of photoresist layer on the resist underlayer film; and
a development step of irradiating a predetermined region of the photoresist layer with radiation for development, thereby obtaining a resist pattern.

13. A circuit pattern formation method, comprising:
a resist underlayer film formation step of forming a resist underlayer film on a substrate using the film forming composition for lithography according to claim 1;
an intermediate layer film formation step of forming an intermediate layer film on the resist underlayer film;
a photoresist layer formation step of forming at least one layer of photoresist layer on the intermediate layer film;
a resist pattern formation step of irradiating a predetermined region of the photoresist layer formed through the photoresist layer formation step with radiation for development, thereby obtaining a resist pattern;
an intermediate layer film pattern formation step of etching the intermediate layer film with the resist pattern as a mask, thereby obtaining an intermediate layer film pattern;
a resist underlayer film pattern formation step of etching the resist underlayer film with the intermediate layer film pattern as a mask, thereby obtaining a resist underlayer film pattern; and
a substrate pattern formation step of etching the substrate with the resist underlayer film pattern as a mask, thereby obtaining a substrate pattern.
